(19) United States
(12) Patent Application Publication
(10) Pub. No.: US 2004/0039211 A1
(43) Pub. Date: Feb. 26, 2004

(54) Tetrapyrroles

(75) Inventors: Irwin Fridovich, Durham, NC (US); Ines Batinic-Haberle, Durham, NC (US); Ivan Spasojevic, Durham, NC (US); James D. Crapo, Englewood, CO (US); Brian J. Day, Englewood, CO (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY AND NATIONAL JEWISH MEDICAL AND RESEARCH CENTER

(21) Appl. No.: 10/424,150

(22) Filed: Apr. 28, 2003

Related U.S. Application Data
(63) Continuation of application No. 09/880,075, filed on Jun. 14, 2001, now abandoned.
(60) Provisional application No. 60/211,875, filed on Jun. 14, 2000.

(51) Int. Cl. C 07D 4 3/14; C07H 17/02

(52) U.S. Cl. 548/518; 536/17.4

(57) ABSTRACT

The present invention relates, in general, to a method of modulating physiological and pathological processes and, in particular, to a method of modulating cellular levels of oxidants and thereby processes in which such oxidants are a participant. The invention also relates to compounds and compositions suitable for use in such methods.

This application claims priority from Provisional Application No. 60/211,875, filed Jun. 14, 2000, the entire content of which is incorporated herein by reference.

### TECHNTCAL FIELD

The present invention relates, in general, to a method of modulating physiological and pathological processes and, in particular, to a method of modulating cellular levels of oxidants and thereby processes in which such oxidants are a participant. The invention also relates to compounds and compositions suitable for use in such methods.

### BACKGROUND

Oxidants are produced as part of the normal metabolism of all cells but also are an important component of the pathogenesis of many disease processes. Reactive oxygen species, for example, are critical elements of the pathogenesis of diseases of the lung, the cardiovascular system, the gastrointestinal system, the central nervous system, immune system and skeletal muscle. Oxygen free radicals also play a role in modulating the effects of nitric oxide (NO.). In this context, they contribute to the pathogenesis of vascular disorders, inflammatory diseases, autoimmunity, cancer and the aging process.

A critical balance of defensive enzymes against oxidants is required to maintain normal cell and organ function. Superoxide dismutases (SODs) are a family of metalloenzymes that catalyze the intra- and extracellular conversion of O₂⁻ into H₂O₂ plus O₂, and represent the first line of defense against the detrimental effects of superoxide radicals. Mammals produce 3 distinct SODs. One is a dimeric copper- and zinc-containing enzyme (CuZn SOD) found in the cytosol of all cells, the second is a tetrameric manganese-containing SOD (Mn SOD) found in the matrix space of mitochondria, and the third is a tetrameric, glycosylated, copper- and zinc-containing enzyme (EC-SOD) found in the extracellular fluids and bound to the extracellular matrix. Several other important antioxidant enzymes are known to exist within cells, including catalase and glutathione peroxidase. While extracellular fluids and the extracellular matrix contain only small amounts of these enzymes, other extracellular antioxidants are also known to be present, including radical scavengers and inhibitors of lipid peroxidation, such as ascorbic acid and uric acid (Halliwell et al. Arch. Biochem. Biophys. 280:1 (1990)).

The present invention relates generally to low molecular weight tetrapyrroles suitable for use in modulating intra- and extracellular processes in which partially reduced oxygen species, for example, superoxide radicals, or other oxidants such as hydrogen peroxide, peroxynitrite or lipid peroxide, are participants. The compounds and methods of the invention find application in various physiologic and pathologic processes in which oxidative stress plays a role.

### SUMMARY OF THE INVENTTON

The present invention relates to a method of modulating intra- or extracellular levels of oxidants such as superoxide radicals, peroxynitrite, hydroxyl radicals and thiyl radicals. More particularly, the invention relates to a method of modulating normal or pathological processes involving superoxide radicals, nitric oxide or peroxynitrite using low molecular weight antioxidants, and to substituted tetrapyrroles suitable for use in such a method.

Objects and advantages of the present invention will be clear from the description that follows.

### BRIEF DESCRIPTION OF THE DRAWING

FIG. 1. Structures of compounds of Formula I and II the invention.

FIGS. 2A-2D. FIG. 2A. The schematic structure of biliverdin dimethylester in its “open” keto form. H₃BVMDE. FIG. 2B. The schematic structure of biliverdin dimethylester in its “closed” enol form, H₃BVMDE. FIG. 2C. The dimeric manganese(III) complex, {Mn^{III}BVDME}₂. FIG. 2D. The Chem-3D presentation of the {Mn^{III}BVDME}₂ wherein pyrrolic subtituents are omitted for clarity.

FIG. 3. The absorbances at 390 nm, 362 nm, and 898 nm are plotted vs concentration of {Mn^{III}BVDME}₂ in the methanol in the range 2×10⁻⁸ M to 8×10⁻⁵ M. The data are obtained in 1 cm and 10 cm spectrophotometric cells, but are presented as though all were obtained in a 10 cm cell.

FIG. 4. The formation of the {Mn^{III}BVDME}₂ at 25° C. at 1:1, metal to ligand ratio (20 μM MnCl₂ and 20 μM BVDME³⁻) in 90/10 (v/v) methanouaqueous solution, pH*7.4, 0.05 M tris buffer. Inset: The absorbance of biliverdin methylester at 668.4 nm vs pH*, 90/10 methanol/aqeous solution, 0.01 M tris and Pipes buffer.

FIGS. 5A and 5B. FIG. 5A. Electrospray mass spectrometry of 300 μM {Mn^{III}BVDME}₂ in methanol at cone voltages of 30 V (A), 90 V (B) and 150 V (C). FIG. 5B. The ion intensities of the major peaks as a function of cone voltage: circles, protonated ligand H₄BVDME⁺; diamonds, oxidized dimer {Mn^{IV}BVDME⁺,Mn^{III}BVDME}; triangles up, cationic dimer {Mn^{III}BVDME,Mn^{III},XBVDME⁺}, includes species that have X as a proton, sodium and potassium; squares, cationic monomer, Mn^{III}HBVDME⁺; triangles down, oxidized monomer, Mn^{IV}BVDME⁺.

FIGS. 6A and 6B. Freezing point measurements of pure bromoform and bromoform in the presence of 16.8 mM manganese(III) biliverdin dimethylester and 17.3 mM 4,4 bipyridyl. Three independent measurements were performed, FIG. 6A, and are averaged for clarity in the shorter period of time in FIG. 6B.

FIG. 7. Magnetic susceptibility measurement of the solid {Mn^{III}BVDNE}₂. The linear relationship between χ_{g} and 1/(T+27.1) is in accord with Curie-Weiss law, eq 3.

FIG. 8. Cyclic voltammogram of {Mn^{III}BVDME}₂ and BVDME³⁻ in 90/10 (v/v) MeOW/H₂O solution, pH 7.9 (0.1 M NaCl), scan rate 0.1 V/s.

FIG. 9. Fixed potential chronocoulometric measurements of 0.5 mM {Mn^{III}BVDME}₂ and Mn^{III}TM-2-PyP⁵⁺ in a 90/10 (v/v) MeOH/ H₂O, pH* 7.9 (0.1 M NaCl).

FIG. 10. Cyclic voltammogram of 0.5 mM {Mn^{III}BVDME}₂ in a 90/10 MeOH/H₂O, pH* 5.8 and 7.9, scan rate 2 V/s (0.1 M NaCl).

FIG. 11. Cyclic voltammogram of 0.5 mM {Mn^{III}BVDME}₂ in a 90/10 MeOH/H₂O, pH* 7.9 and 10.0, scan rate 3 V/s (0.1 M NaCl).

FIG. 12. The plot of {(v₀/vᵢ)−1} vs the concentration of {Mn^{III}BVDME}₂ expressed per manganese. The v₀ is the rate of reduction of 10 μM cytochrome c by O₂. The vᵢ is the rate of reduction of cytochome c inhibited by the porphyrin catalyst in the presence of 0.1 mM EDTA in 0.05 M tris buffer at pH 7.8. 40 μM xanthine. ˜2 nM xanthine oxidase at 25° C. The total volume of the assay solution is 3 mL.

FIG. 13. Growth curves of SOD-proficient AB1157 (circles) and SOD-deficient E. coli JI132 in the presence (13 μM) (diamonds) and absence of {Mn^{III}BVDME}₂ (triangle down) in minimal medium (five amino acids) under aerobic conditions, pH 7.8. The 20 mM ethanolic/albumin solution of compound was diluted into the medium. Also the growth of SOD-deficient E. coli was followed in the presence of 0.15% ethanol only (squares).

FIG. 14. Structure of manganese (III) bilirubin ditaurate.

### DETAILED DESCRIPTION OF THE INVENTION

The present invention relates to methods of protecting against the deleterious effects of oxidants, particularly, superoxide radicals, and peroxynitrite, and to methods of preventing and treating diseases and disorders that involve or result from oxidant stress. The invention also relates to methods of modulating biological processes involving oxidants, including superoxide radicals, nitric oxide and peroxynitrite. The invention further relates to compounds and compositions, including low molecular weight antioxidants (eg mimetics of scavengers of reactive oxygen species, including mimetics of SODs and catalases) and formulations thereof, suitable for use in such methods.

Mimetics of scavengers of reactive oxygen species appropriate for use in the present methods include substituted tetrapyrroles, or pharmaceutically acceptable salts thereof. The invention includes both metal-free and metal-bound tetrapyrroles, preferably those where the metal ion allows formation of a dimeric cyclic structure. Manganese derivatives are preferred, however, metals other than manganese can be used, for example iron. The five-coordination of the manganese in a dimeric environment increases the specificity of the metal site for the O₂⁻ since NO⁻ and H₂O₂ as well as other ligand binding is restricted. For that same reason the otherwise facile axial ligation of iron metal centers is avoided. Consequently, the toxicity of the iron compound due to its interaction with amino acid residues is diminished. Co(III/II), Zn(II), Cu(I/II) and Ni(II) can also be used as the metal center in tetrapyrrole complexes.

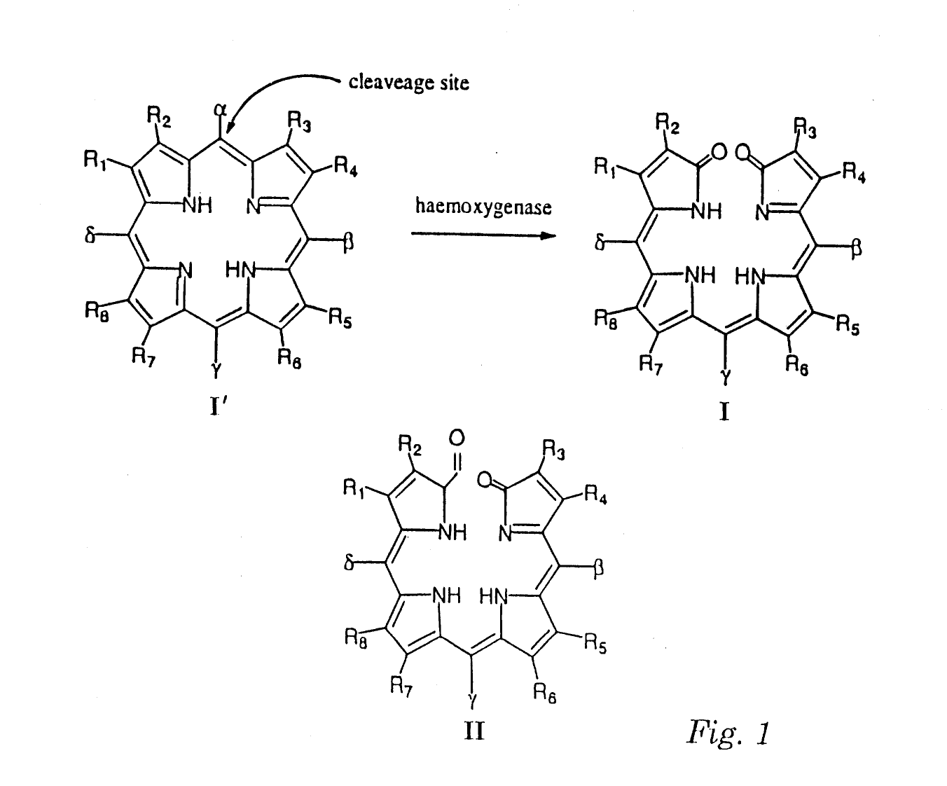

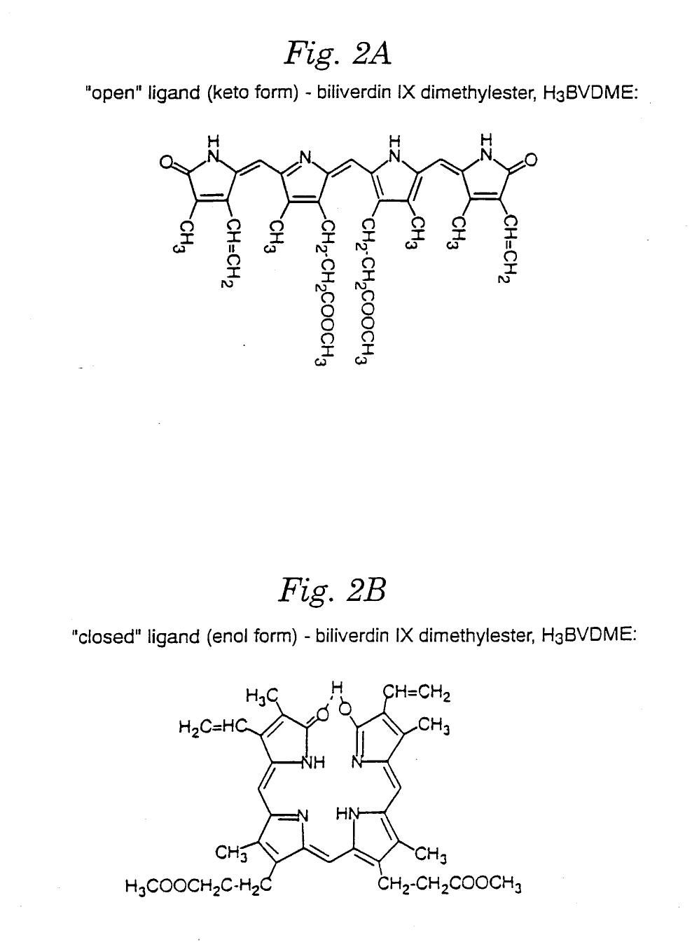

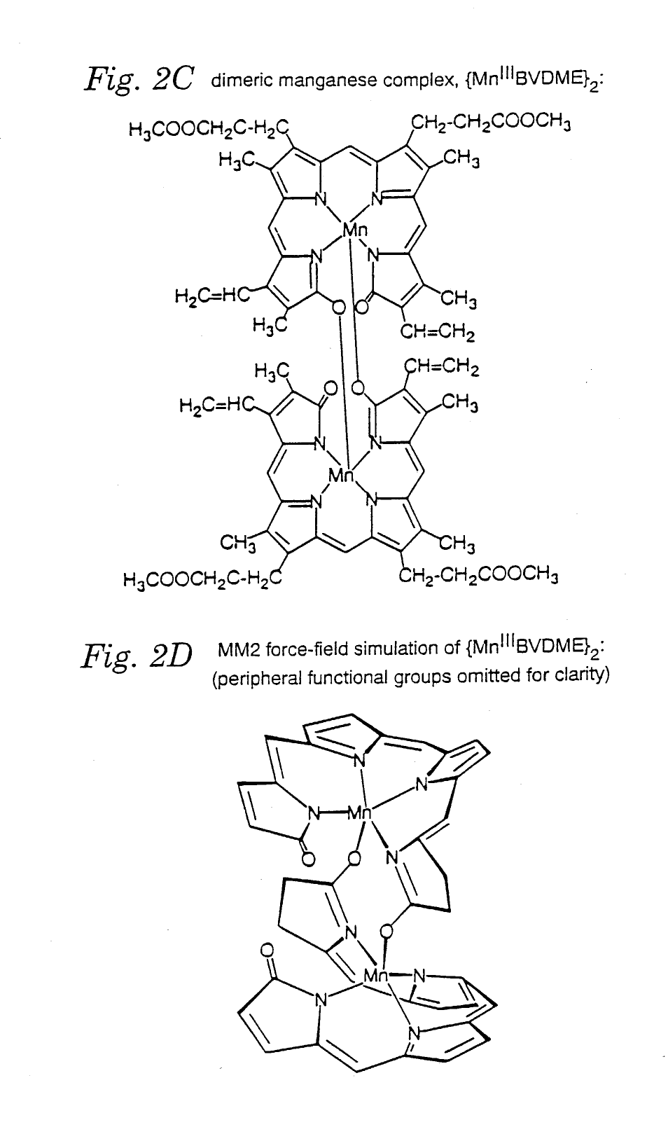

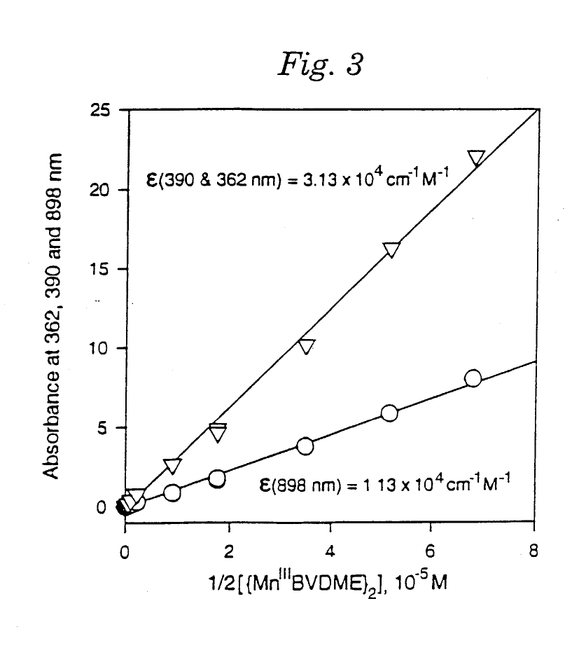

The mimetics of the present invention are shown in FIG. 1 and include pharmaceutically acceptable salts thereof and dimeric forms thereof (see, for example, FIG. 2C). The mimetics of the present invention can be of Formula I, which depicts derivatives of biliverdin in keto form, or can be of Formula II, which depicts derivatives of formylbiliverdin (Fuhrhop et al, Liebigs, Ann. Chem. 1450-1466 (1974)). Biliverdin is formally derived from protoporphyrin-IX by oxidative removal of one α-carbon linkage (see FIG. 1). All naturally occuring bile pigments are therefore assumed to IXα in that the arrangement of the β substituents corresponds to that in biliverdin (O'Carra et al, J. Chromatog. 50:458-468 (1970), Gray et al, J. Chem. Soc. 3085-3099 (1958)). The present invention includes all related compounds that have one or more of the biliverdin groups substituted, including mesoporphyrin IX (vinyl groups reduced to ethyl), hematoporphyrin LX (vinyl groups substituted by α-hydroxyethyls), deuteroporphyrin 1×(no substituent on 2 and 4 beta positions), octaethylbiliverdin (all beta substituents are ethyl groups), etioporphynrn (one ethyl and one methyl group on each pyrrolic unit). (See Table 1 of FIG. 1.) The invention also includes derivatives of bilirubins that are the reduced form of the compounds of Formulas I and II. Such compounds are reduced at methine bridges β, γ and δ in Formulas I and II. The invention further includes the derivatives of the mimetics disclosed in application Ser. No. 08/663,028, application Ser. No. 09/296,615 and application Ser. No. 09/184,982 that can undergo oxidative cleavage with ascorbic acid/On system followed by KOH and BF₃/MeOH treatment (Bonnet et al. J. Chem. Soc. Chem. Commun. 237-238 (1970)) (where no meso substituents are present). When there are phenyls, or substituted phenyls, or pyridyls, or substituted pyridyls, or other substituents, at meso positions, cleavage can be accomplished by treating the porphyrins with thallium (IV) and cerium (IV) salts (Evans et al. J. Chem. Soc. Perkin. Trans. 768-773 (1978)).

With reference to Formulas I and II of FIG. 1:

R₁ through R₈ are, independently, —H, alkyl, 2-hydroxyalkyl, methoxyalkyl, halogen, nitro, cyano, trialkylammonium, formyl, amide of carboxylic acid, alkyl ester of carboxylic acid, carboxylic acid, glucuronyl or glyceryl ester of carboxylic acid, 1,2-dihydroxyalkyl, acetyl, vinyl, glycosyl or, taurate, and

β, γ and δ are, independently, —H, acetyl, glycyl, benzoate, phenylsulfonate, 2-, or 3-, or 4-N-alkyl-pyridyl, nitrophenyl, halophenyl, methoxyalkyl, halogen, nitro, cyano, trialkylammonium, formyl, amide of carboxylic acid.

Preferably, R₁ through R₈ are, independently, —H, C₁-C₅ alkyl, 2-hydroxy C₁-C₅ alkyl, C₁-C₅alkyl ester of C₁-C₅carboxylic acid, C₁-C₅carboxylic acid, glucuronyl or glyceryl ester of C₁-C₅carboxylic acid, 1,2-dihydroxy C₁-C₅ alkyl, acetyl, vinyl, glycosyl, taurate, chloro, fluoro, bromo, nitro, cyano, trimethylammonium, or formyl, and

β, γ and δ are, independently, —H, acetyl, glycyl, benzoato, phenylsulfonato, 2-, 3- or 4-N-C₁-C₅ alkyl-pyridyl, nitrophenyl, bromo-, chloro- or fluorophenyl or 2-, 3- or 4-N-C₁-C₅alkylsulfonatopyridyl.

Specific examples of mimetics of the invention are shown in FIG. 1, with reference to Formulas I and II and to the R₁-R₈ and β, γ and δ substituents shown in Table 1 in the context of Compound I′, and in FIG. 2. The compound of FIG. 2C is a particularly preferred compound. The dimeric structure shown by FIG. 2C is important for giving rise to the favorable metal-centered redox potential, thus to the powerful SOD mimetic activity with k_{cat}=5.5×10⁷ M⁻¹ s⁻¹. The compound of FIG. 2C has a specific activity of 10,700 units/mg, about three times as high as that of the enzyme itself. The specific nature of the particular dimeric structure is that it allows the stabilization of the +4 metal oxidation state. This is achieved through the fifth coordination of each manganese of one biliverdin subunit to the enolic oxygen of the other subunit. Thus, as a consequence of the unique features of the compound, the dismutation of O₂⁻ is catalyzed by Mn(III)/Mn(IV) redox couple whose E_{1/2}=+0.45 V vs NHE in water. The Mn(III)/Mn(II) couple is at E_{1/2}=−0.23 V vs NIE. The latter potential is too negative to allow the dismutation of O₂⁻. This is the first compound reported that utilizes a metal (III)/(IV) couple for the O₂⁻ dismutation rather than Mn(III)/Mn(II) couple as do all the manganese-based SOD mimetics reported to date. Moreover the dimeric environment restricts the reactivity of the compound towards NO and H₂O₂ enhancing its SOD-like specificity. Accordingly, the H₂O₂ induced degradation of the compound occurs 4 orders of magnitude slower than for manganese(III) tetrakis(N-methylpyridinium-2-yl)porphyrin with k=6.4×10⁻⁴ M⁻¹ s⁻¹. No reactivity towards NO has been detected. At 20 μM the compound is stable in the presence of 900-fold excess of EDTA at pH 7.8, thus would resist biological chelators. The compound showed significant protection of SOD-deficient E. coli when crowing under aerobic conditions. This bacterial model has been previously proven to predict the potency of the compounds in rodents model of diseases.

The manganese biliverdin dimethyl ester has previously been reported by Fuhrhop (Liebigs. Ann. Chem. 1131 (1975)). However, a monomeric, phlorin-type (porphyrin-type) of structure was suggested where enolic proton was hydrogen-bonded to the keto oxygen. That structure would allow the existence only of the Mn(III)/Mn(II) redox, which, due to the electron-donating groups on the pyrrole groups of the biliverdin would be too negative in potential to allow any significant SOD-like activity. Moreover the Mn +2 oxidation state was suggested as a resting state, whereas the actual valence is Mn +3.

Where isomers of compounds of Formula I and II (and dimeric forms thereof) are possible, all such isomers of the herein described mimetics are within the scope of the invention.

Mimetics preferred for use in the present methods can be selected by assaying for SOD or catalase activity. Mimetics can also be screened for their ability to scavenge ONOO⁻ (as determined by the method of Szabo et al, FEBS Lett. 381:82 (1996)).

SOD activity can be monitored in the presence and absence of EDTA using the method of McCord and Fridovich (J. Biol. Chem. 244:6049 (1969)). The efficacy of a mimetic can also be determined by measuring the effect of the mimetic on the aerobic growth of a SOD null E. coli strain versus a parent strain. Specifically, parental E. coli (AB1157) and SOD null E. coli. (JI132) can be grown in M9 medium containing 0.2% casamino acids and 0.2% glucose at pH 7.0 and 37° C., growth can be monitored in terms of turbidity followed at 700 nm. This assay can be made more selective for SOD mimetics by omitting the branched chain, aromatic and sulphur-containing amino acids from the medium (glucose minimal medium (M9), plus 5 essential amino acids).

Efficacy of active mimetics can also be assessed by determining their ability to protect mammalian cells against methylviologen (paraquat)-induced toxicity. Specifically, rat L2 cells grown as described below and seeded into 24 well dishes can be pre-incubated with various concentrations of the SOD mimetic and then incubated with a concentration of methylviologen previously shown to produce an LC₇₅ in control L2 cells. Efficacy of the mimetic can be correlated with a decrease in the methylviologen-induced LDH release (St. Clair et al. FEBS Lett. 293:199 (1991)).

The efficacy of SOD mimetics can be tested in vivo with mouse and/or rat models using both aerosol administration and parenteral injection. For example, male Balb/c mice can be randomized into 4 groups of 8 mice each to form a standard 2×2 contingency statistical model. Animals can be treated with either paraquat (40 mg/kg, ip) or saline and treated with SOD mimetic or vehicle control. Lung injury can be assessed 48 hours after paraquat treatment by analysis of bronchoalveolar lavage fluid (BALF) damage parameters (LDH, protein and % PMN) as previously described (Hampson et al, Tox. Appl. Pharm. 98:206 (1989); Day et al, J. Pharm. Methods 24:1 (1990)). Lungs from 2 mice of each group can be instillation-fixed with 4% paraformaldehyde and processed for histopathology at the light microscopic level.

Catalase activity can be monitored by measuring absorbance at 240 nm in the presence of hydrogen peroxide (see Beers and Sizer, J. Biol. Chem. 195:133 (1952)) or by measuring oxygen evolution with a Clark oxygen electrode (Del R10 et al, Anal. Biochem. 80:409 (1977)).

The ability of mimetics to inhibit lipid peroxidation can be assessed as described by Day et al, Free Rad. Biol. Med. 26:730 (1990). Iron and ascorbate can be used to initiate lipid peroxidation in tissue homogenates and the formation of thiobarbituric acid reactive species (T BARS) measured.

Active mimetics can be tested for toxicity in mammalian cell culture by measuring lactate dehydrogenase (LDH) release. Specifically, rat L2 cells (a lung Type II like cell (Kaighn and Douglas. J. Cell Biol. 59: 160a (1973)) can be grown in Ham's F-1 medium with 10% fetal calf serum supplement at pH 7.4 and 37° C.; cells can be seeded at equal densities in 24 well culture dishes and grown to approximately 90% confluence: SOD mimetics can be added to the cells over a broad range of concentrations (eg micromolar doses in minimal essential medium (MEM)) and incubated for 24 hours. Toxicity can be assessed by morphology and by measuring the release of the cytosolic injury marker. LDH (eg on a thermokinetic plate reader), as described by Day et al (J. Pharmacol. Exp. Ther. 275:1227 (1995); oxidation of NADH is measured at 340 nm).

The mimetics of the present invention are suitable for use in a variety of methods. The compounds of Formulas I and II, particularly the dimeric metal bound forms thereof (advantageously, the manganese bound forms), are characterized by the ability to inhibit lipid peroxidation. Accordingly, these compounds are preferred for use in the treatment of diseases or disorders associated with elevated levels of lipid peroxidation. The compounds are further preferred for use in the treatment of diseases or disorders mediated by oxidative stress. Inflammatory diseases are examples, including asthma, inflammatory bowel disease, diabetes, arthritis and vasculitis.

The compounds of the invention (advantageously, dimeric metal bound forms thereof) can also be used in methods designed to regulate NO. levels by targeting the above-described porphinoids to strategic locations. NO. is an intercellular signal and, as such, NO. must traverse the extracellular matrix to exert its effects. NO., however, is highly sensitive to inactivation mediated by O₂⁻ present in the extracellular spaces. The substituted tetrapyrroles of the invention can increase bioavailability of NO. by preventing its degradation by O₂⁻.

The mimetics of the invention (particularly, dimeric metal bound forms thereof) can also be used as catalytic scavengers of reactive oxygen species to protect against ischemia reperfusion injuries associated with myocardial infarction, coronary bypass surgery, stroke, acute head trauma, organ reperfusion following transplantation, bowel ischemia, hemorrhagic shock, pulmonary infarction, surgical occlusion of blood flow, and soft tissue injury. The mimetics (particularly, dimeric metal bound forms) can further be used to protect against skeletal muscle reperfusion injuries. The mimetics (particularly, dimeric metal bound forms) can also be used to protect against damage to the eye due to sunlight (and to the skin) as well as glaucoma, cataract and macular degeneration of the eye. The mimetics (particularly, dimeric metal bound forms) can also be used to treat burns and skin diseases, such as dermatitis, psoriasis and other inflammatory skin diseases. Diseases of the bone are also amenable to treatment with the mimetics. Further, connective tissue disorders associated with defects in collagen synthesis or degradation can be expected to be susceptible to treatment with the present mimetics (particularly, dimeric metal bound forms), as should the generalized deficits of aging. Liver cirrhosis and renal diseases (including glomerular nephritis, acute tubular necrosis, nephroderosis and dialysis induced complications) are also amenable to treatment with the present mimetics (particularly, dimeric metal bond forms thereof).

The mimetics of the invention (particularly, dimeric metal bound forms) can also be used as catalytic scavengers of reactive oxygen species to increase the very limited storage viability of transplanted hearts, livers, lungs, kidneys, skin and other organs and tissues. The invention also provides methods of inhibiting damage due to autoxidation of substances resulting in the formation of O₂⁻ including food products, pharmaceuticals, stored blood, etc. To effect this end, the mimetics of the invention are added to food products, pharmaceuticals, stored blood and the like, in an amount sufficient to inhibit or prevent oxidation damage and thereby to inhibit or prevent the degradation associated with the autoxidation reactions. (For other uses of the mimetics of the invention, see U.S. Pat. No. 5,227,405). The amount of mimetic to be used in a particular treatment or to be associated with a particular substance can be determined by one skilled in the art.

The mimetics (particularly, dimeric metal bound forms) of the invention can also be used to scavenge peroxynitrite as a negatively charged peroxynitrite anion can compete with negatively charged enolate for the 5^{th} coordination site of the manganese.

Further examples of specific diseases/disorders appropriate for treatment using the mimetics of the present invention, advantageously, dimeric metal bound forms, include diseases of the cardiovascular system (including cardiomyopathy, ischemia and atherosclerotic coronary vascular disease), central nervous system (including AIDS dementia, stroke, amyotrophic lateral sclerosis (ALS), Parkinson's disease and Huntington's disease) and diseases of the musculature (including diaphramatic diseases (eg respiratory fatigue in chronic obstructive pulmonary disease, cardiac fatigue of congestive heart failure, muscle weakness syndromes associated with myopathies. GES and multiple sclerosis). Many neurologic disorders (including epilepsy, stroke, Huntington's disease, Parkinson's disease, ALS, Alzheimer's and AIDS dementia) are associated with an over stimulation of the major subtype of glutamate receptor, the NMDA (or N-methyl-D-aspartate) subtype. On stimulation of the NMDA receptor, excessive neuronal calcium concentrations contribute to a series of membrane and cytoplasmic events leading to production of oxygen free radicals and nitric oxide (NO.). Interactions between oxygen free radicals and NO. have been shown to contribute to neuronal cell death. Well-established neuronal cortical culture models of NMDA-toxicity have been developed and used as the basis for drug development. In these same systems, the mimetics of the present invention inhibit NMDA-induced injury. The formation of O₂⁻ radicals is an obligate step in the intracellular events culminating in excitotoxic death of cortical neurons and further demonstrate that the mimetics of the invention can be used to scavenge O₂⁻ radicals and thereby serve as protectants against excitotoxic injury.

The present invention also relates to methods of treating AIDS. The Nf Kappa B promoter is used by the HIV virus for replication. This promoter is redox sensitive, therefore, an oxidant can regulate this process. This has been shown previously for two metalloporphyrins distinct from those of the present invention (Song et al, Antiviral Chem. and Chemother. 8:85 (1997)). The invention also relates to methods of treating systemic hypertension, atherosclerosis, edema, septic shock, pulmonary hypertension, including primary pulmonary hypertension, impotence, infertility, endometriosis, premature uterine contractions, microbial infections, gout, cancer and in the treatment of Type I or Type II diabetes mellitus. The mimetics of the invention (particularly, dimeric metal bound forms) can be used to prevent injury to pancreatic islet cells and therefore prevent or delay onset of symptoms of diabetes mellitus. In a similar manner, the mimetics of the invention can be used to ameliorate inflammatory or oxidative injury to the pancreas and in the prevention and treatment of pancreatitis. The mimetics of the invention (particularly, dimeric metal bound forms) can be used to ameliorate the toxic effects associated with endotoxin, for example, by preserving vascular tone and preventing multi-organ system damage.

As indicated above, inflammations, particularly inflammations of the lung, are amenable to treatment using the present mimetics (particularly, dimeric metal bound forms) (particularly the inflammatory based disorders of emphysema, asthma, ARDS including oxygen toxicity, pneumonia (especially AIDS-related pneumonia), cystic fibrosis, chronic sinusitis, arthritis and autoimmune diseases (such as lupus or rheumatoid arthritis)). Pulmonary fibrosis and inflammatory reactions of muscles, tendons and ligaments can be treated using the present mimetics (particularly, dimeric metal bound forms thereof). EC-SOD is localized in the interstitial spaces surrounding airways and vasculature smooth muscle cells. EC-SOD and O₂⁻ mediate the antiinflammatory—proinflammatory balance in the alveolar septum. NO. released by alveolar septal cells acts to suppress inflammation unless it reacts with O₂⁻ to form ONOO⁻. By scavenging O₂⁻ EC-SOD tips the balance in the alveolar septum against inflammation. Significant amounts of ONOO⁻ will form only when EC-SOD is deficient or when there is greatly increased O₂⁻ release. Mimetics described herein can be used to protect against destruction caused by hyperoxia.

The invention further relates to methods of treating memory disorders. It is believed that nitric oxide is a neurotransmitter involved in long-term memory potentiation. Using an EC-SOD knock-out mouse model (Carlsson et al, Proc. Natl. Acad. Sci. USA 92:6264 (1995)), it has been shown that learning impairment correlates with reduced superoxide scavenging in extracellular spaces of the brain. Reduced scavenging results in higher extracellular O₂⁻ levels. O₂⁻ is believed to react with nitric oxide thereby preventing or inhibiting nitric oxide-mediated neurotransmission and thus long-term memory potentiation. The mimetics of the invention, particularly, dimeric metal bound forms, can be used to treat dementias and memory/learning disorders.

The availability of the mimetics of the invention also makes possible studies of processes mediated by O₂⁻, nitric oxide and peroxynitrite.

The mimetics described above, metal bound and metal free forms, can be formulated into pharmaceutical compositions suitable for use in the present methods. Such compositions include the active agent (mimetic) together with a pharmaceutically acceptable carrier, excipient or diluent and other additives as appropriate (such as solubilizing agents (e.g., glycerol, polyethylene glycol, and dimethylsulfoxide)). A liposome-based composition can also be used. The composition can be present in dosage unit form for example, tablets, capsules or suppositories. Enteric coated tablets and pills, for example, can be used. The composition can also be in the form of a sterile solution suitable for injection or nebulization. Compositions can also be in a form suitable for opthalmic use. The invention also includes compositions formulated for topical administration, such compositions taking the form, for example, of a lotion, cream, gel or ointment. The concentration of active agent to be included in the composition can be selected based on the nature of the agent, the dosage regimen and the result sought.

The dosage of the composition of the invention to be administered can be determined without undue experimentation and will be dependent upon various factors including the nature of the active agent (including whether metal bound or metal free), the route of administration, the patient, and the result sought to be achieved. A suitable dosage of mimetic to be administered IV or topically can be expected to be in the range of about 0.01 to 50 mg/kg/day, preferably, 0.1 to 10 mg/kg/day. For aerosol administration, it is expected that doses will be in the range of 0.001 to 5.0 mg/kg/day, preferably, 0.01 to 1 mg/kg/day. Suitable doses of mimetics will vary, for example, with the mimetic and with the result sought.

Certain aspects of the present invention will be described in greater detail in the non-limiting Examples that follows. As regards preparation of biliverdin dimethylester ligand, attention is directed to Cole et al, Biochemistry 7:2929 (1968), Gray et al, J. Chem. Soc. '264 (1961), Nichol et al, Biochem. Biophys. Acta 177:599 (1969) and Tixier, Ann. Inst. Oceanogr. (Monaco) 22:343 (1945).

### EXAMPLE 1

Experimental Details (Reagents and Methods)

General Reagents. Biliverdin IX dimethylester (H₃BVDME), bilirubin IX (H₄BR) and bilirubin LX dimethylester (H₄BRDME), were obtained from Porphyrin Products (Logan, Utah). Biliverdin IX dimethylester was further recrystallized from chloroform/petroleum ether at 15/50 v/v ratio. Petroleum ether (35-60° C. fraction), chloroform, acetone, diethyl ether (anhydrous), 30% solution of H₂O₂, and methanol were from Mallinckrodt, and dichloromethane was from EM Science, all of highest purity. Manganese(II) acetate (tetrahydrate) (99.99%), manganese(II) chloride (tetrahydrate) (99.99%), sodium L-ascorbate (99+%), ferrocenemethanol (97%), K₃Fe(CN)₆, bromoform (99+%, further redistilled), oxalic acid (99%+) and methyl-d₃ alcohol-d (99.8 atom % D) were from Aldrich. The HCl, KCl, KNO₃, EDTA, glucose, phosphate salts, inorganic salts and KOH were from Mallinckrodt, and casamino acids were from Difco. The volumetric standards, 1.0 M and 0.10 M NaOH and lithium hydroxide (anhydrous) were from Fisher Scientific. The 2-methyl-2-propanol (t-BuOH, 99.5+%), albumin from bovine serum, Pipes disodium salt monohydrate (1,4piperazinebis(ethanesulfonic acid)), succinic acid and xanthine were purchased from Sigma. Deuterium oxide D₂O, 99.9% was from Cambridge Isotope Laboratories. Cytochrome c from horse heart (# 30 396), biliverdin IX dihydrochloride (H₃BVDME×2HCl), (˜80%), 4,4-bipyridyl (99%+), and N,N-bis(salicylidene)ethylenediamine (H₂salen) (99+%) were from Fluka. Xanthine oxidase was prepared by R. D. Wiley and was supplied by K. V. Rajagopalan ((Waud et al, Arch. Biochem. Biophys. 19:695 (1975)). Catalase was from Boehringer. Ammonium oxalate was from J. T. Baker. Ultrapure argon was from National Welders Supply Co, and nitric oxide was from Matheson Gas products. Tris (ultra pure) was from ICN Biomedicals, Inc, NONOate NOC-9 was from CalBiochem and phosphate buffered-saline (PBS buffer) from Life Technologies. Lithium hydrogensuccinate was prepared by neutralizing 1M methanolic solution of succinic acid with 0.5 molar equivalent of LiOH in methanolic solution. Biliverdin and bilirubin 1 mM aqueous stock solutions, pH ˜10 were used throughout work. Elemental analyses were made by Atlantic Vilicrolab, Inc. Norcross, Ga.

Biliverdin IX Dimethylester. The spectral properties of the biliverdin dimethylester ligand, the protonated keto and enol forms of which (Gray et al J. Chem. Soc. 2264 (1961); Bonnet et al, J. Chem. Soc. Chem. Commun. 238 (1970); Nichol et al, Biochim. Biophys. Acta 177:599 (1969); O'Carra et al, J. Chromatog. 50:458 (1970); Chae et al, J. Am. Chem. Soc. 97:4176 (1975)), H₃BVDME are shown in FIGS. 2A and 2B, are in accordance with literature data (Gray et al J. Chem. Soc. 2264 (1961); Bonnet et al, J. Chem. Soc. Chem. Common. 238 (1970); Nichol et al. Biochim. Biophys. Acta 177:599 (1969); O'Carra et al, J. Chromatog. 50:458 (1970); Chae et al, J. Am. Chem. Soc. 97:4176 (1975)). The uv/vis data for the ligand BVDE³⁻ in methanol: ε₆₆₆=1.28×10⁴ cm⁻¹ M⁻¹ and ε₃₇₅=4.45×10⁴ cm⁻¹ M⁻¹. The elemental analysis for H₃BVDME, C₃₅H₃₈N₄O₆: Calcd: C, 68.83%; H, 6.27%: N, 9.17%. Found: C, 68.72%; H, 6.25%; N, 9.17%. The base peak in the electrospray mass spectrum at m/z=611 is assigned to the fully protonated biliverdin dimethylester. H₄BVDME⁺.

Manganese(III) Porphyrins. Mn^{III}TPPCl and Mn^{III}TSPPNa₃ obtained from Mid-Century Chemicals (Chicago, Ill.), and Mn^{III}OEPCl from Aldrich were used as received. Mn^{III}TM(E)-2(4)-PyPCl₅ and Mn^{III}OBTM-4-PyPCl₄ were prepared as described previously (Batinic-Haberle et al. Inorg. Chem. 38:4011 (1999), Batinic-Haberle et al, J. Biol. Chem. 273:24521 (1998), Batinic-Haberle et al, Arch. Biochem. Biophys. 343:225-233 (1997)).

Manganese(III) Salen. The compound was prepared by the published procedure (Boucher, J. lnorg. Nucl. Chem. 36:531 (1974)). The uv/vis in water: ε₂₃₅=3.70×10⁴ cm⁻¹ M⁻¹, ε₂₇₉=1.70×10⁴ cm⁻¹ M⁻¹, ε₃₉₇=4.60×10³ cm⁻M⁻¹. The elemental analysis: Anal. Calcd for Mn^{III}salenCl, C₁₆H₁₄N₂O₂MnCl: C, 53.88; H, 3.96; N; 7.85. Found: C, 54.02; H, 4.06; N, 7.85. The metal-centered redox potential for the Mn(III)/Mn(II) couple, determined as described previously by cyclic voltammetry (Batinic-Haberle et al. Inorg. Chem. 38:4011 (1999)), was found to be E_{1/2}=−0.23 V vs Ag/AgCl in methanol. Based on the E_{1/2} of Mn^{III}TM-2-PyP⁵⁺, ferrocenemethanol and of K₃Fe(CN)₆ in water and methanol, the redox potential of Mn^{III} salen⁺ in water was calculated to be E_{1/2}=−0.13 V vs NHE.

Manganese(III) Biliverdin IX Dimethylester, {Mn^{III}BVDME}₂. The complex was prepared by the modification of the literature methods available for the synthesis of similar compounds (Fuhrhop et al, Liebigs. Ann. Chem. 1131 (1975), Bonnett et a, J. Chem. Soc. Perkin Trans 1,322 (1981)). Accordingly, 50 mg of the recrystallized biliverdin dimethylester was dissolved in a small volume of chloroform and 150 mL of methanol was added. The mixture was heated to ˜60° C. followed by the addition of 15-fold excess of manoanese(II) acetate (0.3 g in 10 mL methanol). The complex formed immediately as evidenced by the disappearance of the absorption at 666 nm and appearance of a band at 898 nm. Most of the solvent was then immediately evaporated on rotary evaporator at room temperature, followed by the addition of 200 mL of chloroform (a little bit of methanol if needed for solubilization) and 200 mL of water to the residue. (Caution—longer exposure at ˜60° C. results in the destruction of the compound.) The mixture was transferred into separatory funnel, and the metal complex was extracted into the chloroform layer leaving the manganese(II) acetate in the aqueous phase. The extraction was repeated thrice. Finally, the chloroform layer was shaken with 5 g of dry MgSO₄ followed by the filtration and evaporation of the solvent almost to dryness. The residue was transferred to an Erlenmeyer flask and dissolved in 4 mL of dichloromethane. Addition of ˜40 mL of petroleum ether gave a precipitate that was collected on a fine fritted glass disc, washed with petroleum ether and dried overnight in vacuo at room temperature. The yield was >80%. The resultant olive-green compound was soluble in ethanol and methanol but was of very low solubility in water (˜10⁻⁸ M). The uv/vis characteristics of manganese(III) biliverdin dimethylester (FIG. 3) are in excellent agreement with the literature data for the similar compounds (Fuhrhop et al, Liebigs. Ann. Cizem. 1131 (1975)). Adherence to the Beer-Lambert law indicates that the same structure exists in the methanolic, pyridine and chloroform solutions in the concentration range of 2×10⁻⁸ M to 8×10⁻⁵ M. The molar absorptivities are calculated per manganese in methanol (FIG. 3): ε₈₉₈=1.13×10⁴ cm¹ M⁻¹, ε₃₉₀=3.13×10⁴ cm⁻¹ M⁻¹ and ε₃₆₂==3.13×10⁴ cm⁻¹ M⁻¹; in pyridine: ε₉₀₈=0.98×10⁴ cm⁻¹ M⁻¹, ε₃₉₃=2.80×10⁴ cm¹ M⁻¹ and ε₃₆₇=2.80×10⁴ cm⁻¹ M⁻¹, and in chloroform: ε₈₉₈=1.07×10⁴ cm⁻¹ M⁻¹, ε₃₉₄=2.80×10⁴ cm⁻¹ M⁻¹ and ε₃₆₇=2.80×10⁴ cm⁻¹ M⁻¹. The elemental analysis for {Mn^{III}BVDME}₂, Mn₂C₇₀H₇₀N₈O₁₂: Calcd: C, 63.44%; H, 5.33%: N, 8.46%. Found: C. 62.78%; H, 5.41%; N, 8.40%. The C/N ratio: Calcd. 8.74. Found, 8.72.

Uv/vis Spectroscopy. The formation and dissociation of {Mn^{III}BVDME}₂ as well as the pH^{*} (pH*-pH is referred to infinitely diluted solution in the 90/10 methanol/aqueous solvent system rather than to infinitely diluted solution in water) titration of the ligand, all in methanouaqueous (90/10, v/v) solution, were studied at 25° C. on UV-2501PC Shimadzu spectrophotometer. Also the effects of EDTA, H₂O₂, and the methanol/water ratio on the stability of the manganese(III) complex were examined. The reactivity of {Mn^{III}BVDME}₂ towards nitric oxide was determined for the reaction with gaseous NO^{*} and with the NONOate NOC-9 in methanol/PBS (50/50, v/v) solution, as described previously (Spasojevic et al, Nitric Oxide: Biology and Chemistry, (2000), in press). Tris and Pipes buffers along with 1.0 M methanol/aqueous (90/10) solution of HCl were used for pH^{*} adjustments in methanol/aqueous solutions. The pH^{*} readings were measured on an Oakton pH-Meter equipped with a Denver Instruments combination glass electrode that was calibrated in methanol/water (90/10, v/v) buffer solutions: 10 mM oxalic acid/10 mM ammonium oxalate (pH^{*}=3.59) and 10 mM succinic acid/lithium hydrogen succinate (pH^{*}=6.55) (De Ligny et al. A. Rec. Trav. Chim. Pays-Bas 79:699 (1960)). When needed the biliverdin and biliverdin dimethylester as well as their reduced forms bilirubin and bilirubin dimethylester were studied.

Electrospray Mass Spectrometry. ESMS measurements were performed on a Micromass-Quattro LC triple-quadrupole mass spectrometer equipped with a pneumatically assisted electrostatic ion source operating at atmospheric pressure. The 600 μM, 60 μM and 6 μM methanol solutions of BVDME³⁻ and ½{Mn^{III}BVDME}₂ solutions were introduced by loop injection into the methanol stream. The mass spectra were acquired in continuum mode, scanning from m/z 600 to 3000 at different cone voltages in the range 30 V-180 V.

Freezing-Point Depression. The measurements were made in a slowly cooled, rubber-foam insulated test tube, in which the bulb of a Normalglas precision thermometer (1/100° C.) was completely immersed into 6 mL of the measured solution. Stirring of the solution was accomplished by the slow eccentric movement of the thermometer provided by a flexible connection of the thermometer to a variable-speed motor.

Magnetic Susceptibility in Solution. Magnetic susceptibilities were determined by the Evans method (Evans, J. Chem. Soc. 2003 (1959)) using 400 MHz Varian NMR spectrometer. Typically ˜2 mg of the compound, dissolved in 0.4 mL D₂O or CD₃OD containing 0.01 M t-BuOH, was placed in a NMR tube along with a capillary that contained the same solvent mixture.

Magnetic Susceptibility in Solid State. Magnetic susceptibility of the solid {Mn^{III}BVDME}₂ was measured on a Faraday balance (Senftle et al, Rev. Sci. Instrum. 29:439 (1958), Thorpe et al, Rev. Sci. Instrum. 30:1006 (1959), Sullivan et al, J. Chem. Educ. 48:345 (1971), Thorpe et al. Coal Geology 36:243 (1998)) automated to record apparent mass changes at programmed temperature and magnetic field intervals. The 5 mg samples were suspended on a Cahn electrobalance with He gas as the heat transfer medium.

Electrochemistry of the Manganese(III) Biliverdin Dimethylester. Measurements were made using a CH Instruments model 600 voltammetric analyzer as described previously(Batinic-Haberle et al, Inorg. Chem. 38:4011 (1999)). A three-electrode system in a small volume (0.5-3.0 mL), with a 3 mm diameter glassy carbon button working electrode (Bioanalytical Systems), Ag/AgCl reference electrode (3 M NaCl, Bioanalytical Systems), and a Pt wire (0.5 mm) as auxiliary electrode, were used. The cyclic votammetry was performed on 0.5 mM (per manganese) methanol/aqueous (90/10, v/v) solutions of the compounds investigated. The ionic strength was kept at 0.10 M (NaCl), and 0.05 M tris buffer was used for pH^{*} adjustment. For calibration purposes, compounds of different hydrophilicities, K₃Fe(CN)₆³⁺, (Kolthof et al, J. Phys. Chem. 39:945 (1935)) ferrocenemethanol and Mn^{III}TM-2-PyP⁵⁺ were studied in both aqueous and methanol/aqueous (90/10, v/v) solutions (I=0.10 M NaCl, 0.05 M tris buffer, pH^{*} 7.9). In all cases the measured E_{1/2} was 170 mV more positive in methanolic than in aqueous solution and this value was used to predict the redox potential in aqueous solution from measurement in methanol/aqueous solution. In addition, Mn^{III}TM-2-PyP⁵⁺ was added as an internal standard when cyclic voltammetry of {Mn^{III}BVDME}₂ in methanol was performed.

The chronocoulometry measurements were performed by recording the change in charge vs time at a fixed potential that was applied to a glassy-carbon button working electrode immersed in 1 mM methanol/aqueous (90/10, v/v) solutions (0.1 M NaCl, 0.05 M tris buffer. pH^{*} 7.9) of ½{Mn^{III}BVDME}₂ and Mn^{III}TM-2-PyP⁵⁺.

SOD Activity. Catalysis of the dismutating of O₂ in vitro was followed by the xanthine oxidase/cytochrome c method (McCord et al, J. Biol. Chem. 244:6049 (1969)).

Effects on E. coli. E. coli strains AB1157 (SOD-proficient, wild type) and JI132 (SOD-deficient, sodAsodB) were obtained from J. A. Imlay (Imlay et al, J. Bacteriol. 169:2967 (1987)). The effect of BVDME³⁻ and {Mn^{III}BVDME}₂ on the growth of these strains was followed aerobically in minimal (five amino acids) medium (Faulkner et al, J. Biol. Chem. 269:23471 (1994)). Cultures were treated as previously described (Faulkner et al, J. Biol. Chem. 269:23471 (1994); Benov et al, J. Biol. Chem. 273:10313 (1998)). Due to the low water solubility, the compounds were solubilized with albumin in medium at a 1:1 molar ratio. Albumin itself had no effect on growth in the concentration range employed. Since methanol was toxic to the SOD-deficient strain of E. coli even when present in medium at 0.15%, the stock solutions of the compounds were prepared in ethanol.

Results

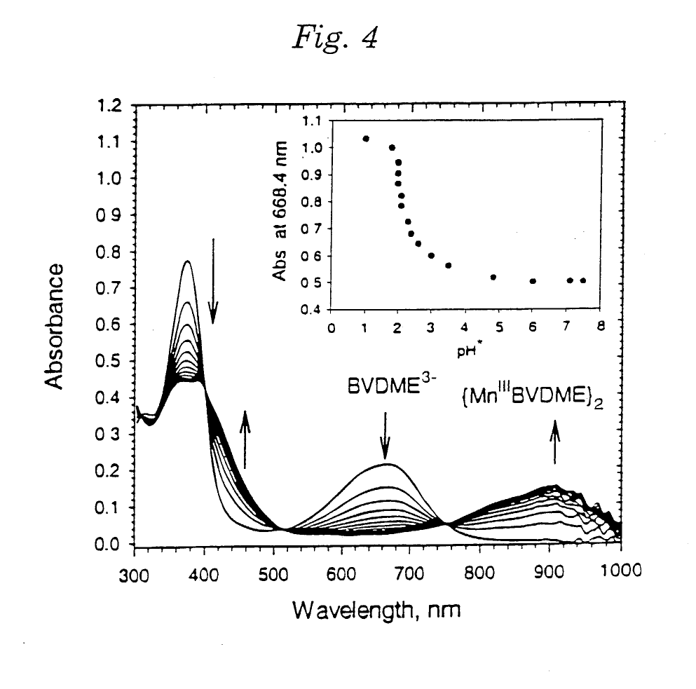

Uv/Vis Spectroscopy. At pH^{*} 7.4 biliverdin dimethylester is in a fully deprotonated form, BVDME³⁻. The data (FIG. 4, inset) showed that protonation of the BVDME³⁻ occurs in the pH^{*} region below 4, which is in agreement with the reported pKa ˜3 (Gray et al, J. Chem. Soc. 2276 (1961); Kuenzle et al, Biochem. J. 130:1147 (1972); O'Carra, Nature 899 (1962)) for the biliverdin, H₄BV⁺. Carboxyl groups are separated from the main chromophores by two methylene groups, so that their ionization is not detectable spectrophotometrically. Thus the spectra of biliverdin and its ester are the same (Gray et al, J. Chem. Soc. 2276 (1961); Kuenzle et al. Biochem. J. 130:1147 (1972); O'Carra, Nature 899 (1962)). At a 1:1, metal to ligand ratio (20 μM), the formation of the complex {Mn^{III}BVDME}₂ at pH^{*} 7.4 (methanouaqueous, 90/10. v/v) goes to completion within 40 min. The multiple isosbestic points (FIG. 4) observed during complex formation indicate only two light-absorbing species in equilibrium, which can be ascribed to fully deprotonated ligand and to the dimeric metal complex. The kinetic trace recorded upon fast mixing of the 30 μM BVDME³⁻ with 75 μM or 150 μM MnCl₂, fits to a single-exponential equation with a zero intercept. This suggests that the rate-limiting step is the formation of the monomeric complex, followed by fast dimerization to yield {Mn^{III}BVDME}₂. The monomeric species was not observable spectrophotometrically. Upon decreasing the pH^{*}, dissociation of the complex occurs. Six isosbestic points were observed only in the pH^{*} region 7.9 to 4.5 where the fully deprotonated ligand is the major species. The spectra obtained resemble the spectra shown in FIG. 4 where complex formation was followed at pH^{*} 7.4. The isosbestic points become obscure at lower pH as a result of multiple ligand protonation.

The same type of manganese(III) complex as {Mn^{III}BVDME}₂, was spectroscopically observed in the reaction of manganese(II) chloride (or acetate) with carboxylate analogue of biliverdin dimethylester, biliverdin (BV³⁻) in 90/10 methanol/aqueous solution, pH^{*} 7.9 (tris buffer), and in water (tris buffer, pH 7.8). However, the complex was not sufficiently stable in the water to permit further characterization.

A very slow hydrolysis of {Mn^{III}BVDME}₂ occurs in 90/10 methanol/aqueous solution, pH^{*} 7.9. In a week, at 25° C., only 14% of the complex dissociated. When the water content was increased to 43%, half the complex dissociated in a week. The 1 μM {Mn^{III}BVDME}₂,was stable in the presence of a 900-fold excess EDTA (0.9 mM) for 2-4 hours at 25° C. in methanol/aqueous (90/10, v/v) solution, 0.05 M tris, pH^{*} 7.9.

At μM concentrations, manganese(III) and iron(III) porphyrins are degraded by H₂O₂ in aqueous solution. pH 7.8 (Batinic-Haberle et al, Inorg. Chem. 38:4011 (1999), Everett et al, J. Trans. Faraday Soc. 49:410 (1953)). Yet, {Mn^{III}BVDME}₂ was extremely resistant to H₂O₂. The reaction was followed in 90/10, v/v methanouaqueous solution at pH^{*} 7.9. In the presence of excess H₂O₂ (6 mM to 120 mM) over {Mn^{III}BVDME}₂ (15 μM and 30 μM), the second-order rate constant k=6.4×10⁻⁴ M⁻¹ s⁻¹ was determined from the linear plot of the observed pseudo-first order rate constants vs [H₂O₂], k_{obs}=k [H₂O₂]. The uv/vis spectroscopy indicated degradation via the reduction of biliverdin to bilirubin followed by loss of metal (Gray et al, J. Chem. Soc. 2276 (1961); Kuenzle et al, Biochem. J. 130:1147 (1972); O'Carra, Nature 899 (1962)). The interaction of biliverdin and bilirubin with H₂O, was also followed in 0.05 M tris buffer, pH 7.8. As observed for its metal complex, biliverdin undergoes reduction by H₂O₂ via bilirubin. Under pseudo-first order conditions (6 mM to 60 mM H₂O₂, 60 μM biliverdin), the second-order rate constant was k=3×10⁻³ M⁻¹ s⁻¹. Bilirubin is more resistant to H₂O₂ than biliverdin or its metal complex. The disappearance of bilirubin was followed at 435 nm (Gray et al, J. Chem. Soc. 2268 (1961)). Under conditions given above and at 60 mM H₂O₂, about 40% of the biliverdin and 14% of bilirubin was degraded within 4.4 hours, and about 14% of the complex was degraded inside 80 min. No change in the spectrum of 14 μM {Mn^{III}BVDME}₂ in the presence of 90-fold excess NO^{*} in 90/10 methanol/PBS was observed after 1 hour.

Electrospray Mass Spectrometry. The dimeric structure of manganese(III) biliverdin dimethylester (FIG. 2) is neutral and can only be observed in ESIMS by association with cations, presumably at enolic oxygens or by metal-centered electrochemical oxidation at the capillary tip (The Electrolytic Nature of Electrospray by Van Berkel. G. J. in Electrospray Ionization Mass Spectrometry, Fundamentals, Instrumentation, And Applications, Cole. B. R., editor. John Wiley & Sons, Inc., New York 1997, pp 65)). Manganese(III) biliverdin dimethylester does not have an easily available Mn(III)/Mn(II) couple, but does have an easily available Mn(III)/Mn(IV) redox. In contrast, the cationic aquamanganese(III) and monohydroxoiron(III) N-alkylpyridyl porphyrins can be easily reduced at the metal centers as a consequence of their positive E_{1/2} (Batinic-Haberle et al, Inorg. Chem. 38:4011 (1999); Batinic-Haberle et al, J. Porphyrins Phthalocyanines 4:217 (2000)). Consequently, their mass spectra contain peaks of reduced species whose intensities depend upon the applied cone voltage.

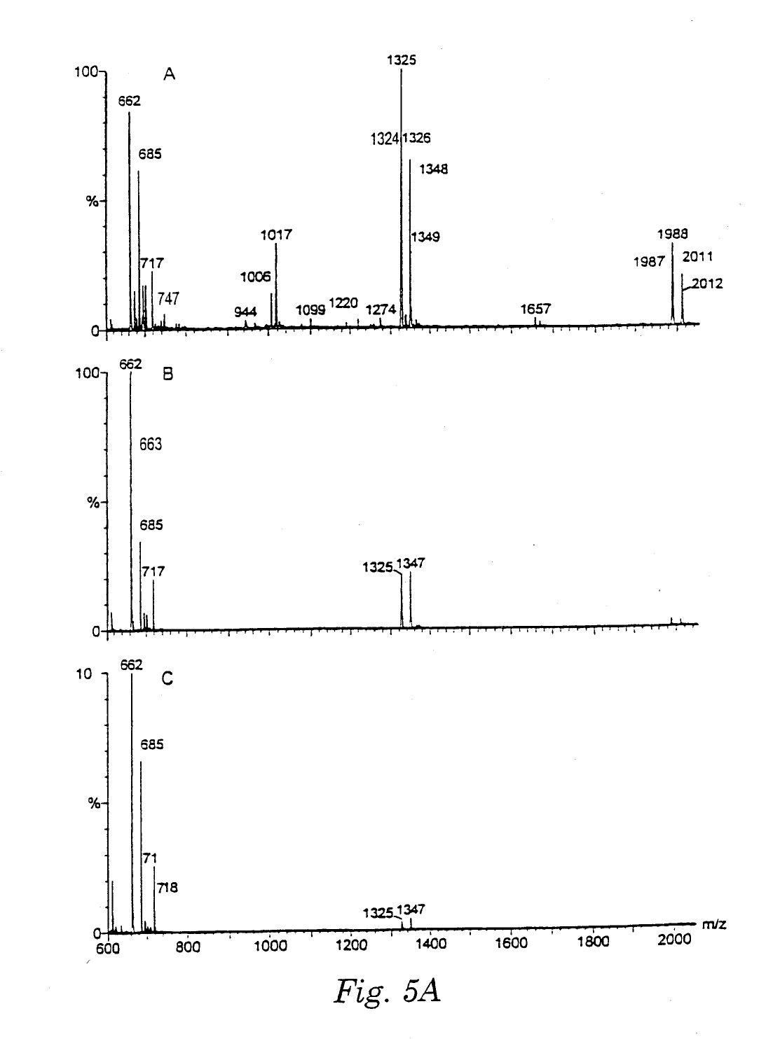

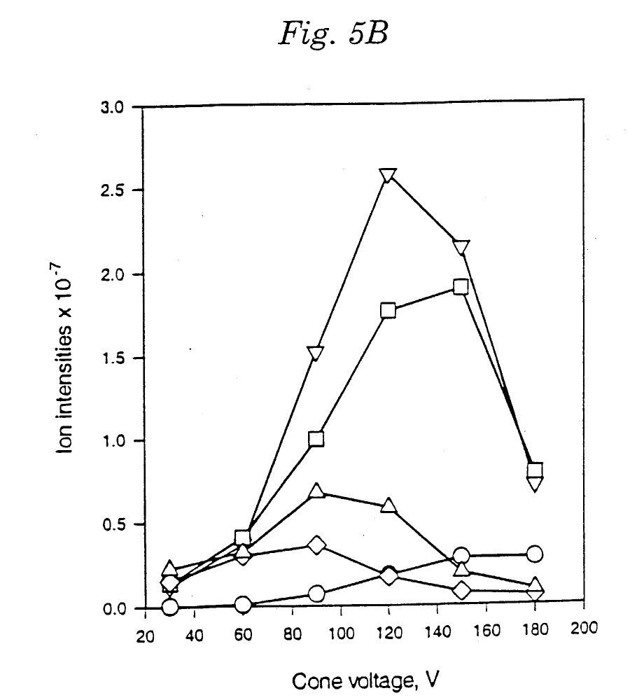

Examples of the ESMS spectra are given in FIG. 5A and the assignment of the peaks in Table 2. In FIG. 5B the intensities of the major ions are given as a function of cone voltage. The isotopic distribution was accounted for in the calculation of ion intensities. At a low cone voltage (30 V) the spectrum shows two major peak groups of similar relative intensities, one around m/z 1325 and the other region around m/z 662. The dominant peak at m/z 1325 is assigned to the protonated dimeric structure {Mn^{III}BVDME,Mn^{III}HBVDME⁺}, and the peak at m/z 1324 to the species oxidized at the metal site, {Mn^{IV}BVDME⁺,Mn^{III}BVDME}. The higher the cone voltage, the lower the intensities of these peaks relative to those ions at m/z 662 and m/z 663 (FIG. 5B). The latter ions are observed as a consequence of the collision induced dissociation of oxidized and protonated dimer, respectively (Table 2). As the cone voltage increases from 30 V to 150 V the relative intensities of the cationic species that bear either a proton, or sodium or potassium increases. Moreover, the higher the cone voltage the higher the ratio of sodiated over protonated species, reflecting the relative stability of the cationic species. Also the ratio of oxidized to cationic dimer decreases, whereas consequently the ratio of oxidized to cationic monomer increases. The dimer persists with the cone voltage as high as 180 V, which is indicative of its high stability, and of its covalent nature. At cone voltage approaching 180 V, more of the ligand becomes detectable at m/z 611. The peaks at m/z 1987 and m/z 1988 may be assigned either to the oxidized and protonated (monomer+dimer) cluster, or to the oxidized or protonated trimer, respectively.

When a methanolic solution of {Mn^{III}BVDME}₂, was diluted 10- and 100 fold, the degree of fragmentation was increased in the same manner as when the cone voltage was elevated. Also, dilution increased the ratio of sodium and potassium over proton, thus increasing the intensities of sodiated and potassiated species. At a 100-fold dilution the relative intensity of the dimeric species is still high. Thus, about 20% of the oxidized and about 20% of the sodiated ion was seen at a cone voltage of 60 V.

In an attempt to understand further the processes underlying the changes in the mass spectrum with cone voltage, selected ions were fragmented in the collision cell. Parent ion m/z 1324 gave m/z 662 as the only product ion, whereas, m/z 1325 gave both m/z 662 and 663. The latter is readily explained since the peak at m/z 1325 is due both to the protonated dimer and to the heavy isotope (¹³C) of the oxidized dimer. Similar experiments with trimeric species gave dimeric and monomeric ions.

Several ions were observed only at lowest (treshold) cone voltage e.g. m/z 674, 693 and 1005. Interestingly, the isotope distribution for these ions was 0.5 m/z indicating that they were doubly charged. Therefore, the m/z 674 is either a cluster of protonated and sodiated monomers or a doubly charged dimer. In either case it is easily collisionally dissociated. Similarly, the doubly charged m/z 1005 is either a cluster of monomer and dimer or trimer. Most of the ions observed at this treshold cone voltage may be explained (Table 2) on the basis of singly and multiply charged ions. The origin of persistent signal at 717 is not known.

Finally, the ESMS behavior of a similar lipophilic porphyrin, chloromanganese(III) octaethylporphyrin, Mn^{III}OEPCl was compared. The base peak was the ion Mn^{III}OEP⁺ at m/z 587, and less than 2% of a dimer was observed at the lowest cone voltage (30 V) for a concentrated (2 mV) solution. This is in marked contrast to {Mn^{III}BVDME}₂ where dimers persist at all cone voltages and dilutions studied.

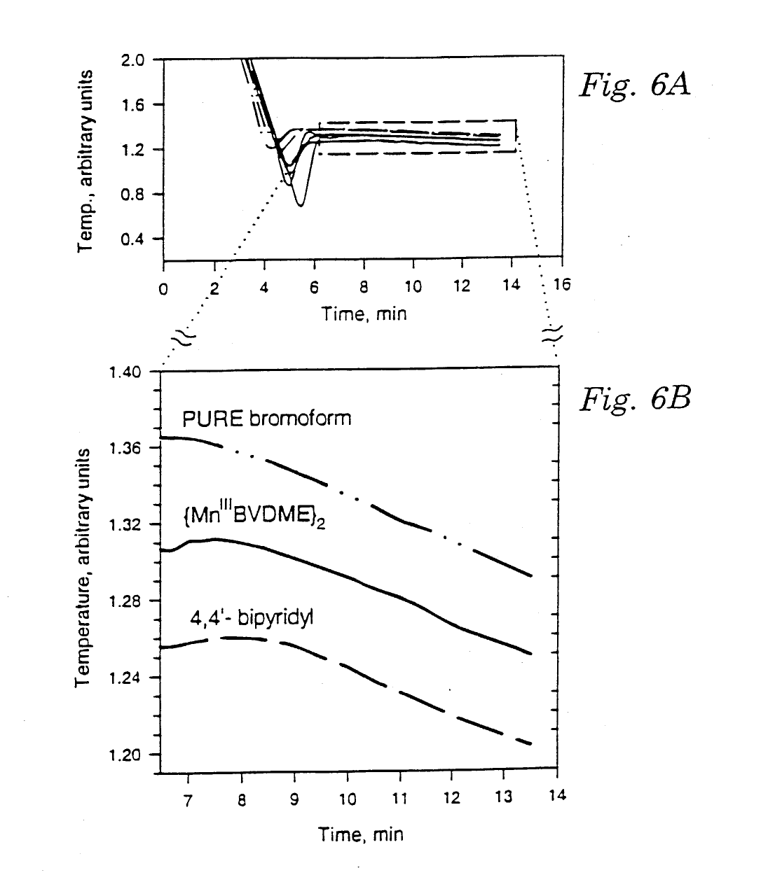

Freezing-point Depression. The measurements were made on 17 mM solutions of 4,4-bipyridyl used as a standard monomeric compound and {Mn^{III}BVDME}₂ (concentration calculated per manganese) in bromoform. Bromoform was chosen since it has large freezing point depression of 14.4° C. m⁻¹ (Handbook of Chemistry and Physics, D. R., Lide, Ed., 74th ed., CRC Press, Boca Raton, 1993-1994; Lange's Handbook of Chemistry, Compiled and Edited by Lange, N. A., 10th ed. McGraw-Hill Book Company, New York 1966)) and is a good solvent for both compounds. Three independent measurements of each compound were made and the data are shown in FIG. 6. Exactly twice the freezing-point depression was observed for the monomeric 4,4-bipyridyl than for equimolar manganese(III) biliverdin dimethyl ester (per manganese concentration), a clear indication of dimeric state of the latter compound.

Magnetic Susceptibility in Solution. The ¹H NMR spectra were obtained independently for both ligands and their manganese complexes at 25° C., permitting the measured susceptibilities to be corrected for the diamagnetic contribution of the ligand. The gram susceptibility, χ_{g} of the compound was calculated using eq 1:

χ_{g}=(3Δv)/(Q2πv₁m)+χₒ [1]

where Q is 2 for the superconducting magnet. Δv is the frequency difference in Hz between the shifted resonance and the t-BuOH reference peak from the capillary insert tube, v₁ is the frequency in Hz of the radio waves generated by the NMR instrument, m is the mass in grams of the compound in 1 mL of the solvent, and χₒ is the mass susceptibility of the solvent, −0.72×10⁻⁶ for the D₂O and −0.66×10⁻⁶ for CD₃OD (Handbook of Chemistry and Physics, D. R., Lide, Ed., 74th ed., CRC Press, Boca Raton, 1993-1994; Lange's Handbook of Chemistry, Compiled and Edited by Lange, N. A., 10th ed., McGraw-Hill Book Company, New York 1966)). The molar susceptibility, χ_{M} was calculated as χ_{M}=χ_{g} M, M being a molar mass. The effective magnetic moments μ_{eff} were then calculated from the eq 2 and are given in BM (Bohr magnetron) in Table 3:

μ_{eff}=2.84√χ_{M}T [2]

Since {Mn^{III}BVDME}₂ is methanol-soluble its magnetic moment was measured in CD₃OD. However, although the solvent effect could be accounted for through term χₒ (eq 1), μ_{eff}=4.57 BM was obtained, which is lower than expected for the +3 oxidation state of manganese. The susceptibility measurements were therefore performed both in deuterated water and methanol for several compounds of known metal oxidation states, but of different charge to hydrophilicity ratios. MnTM(E)-2-PyP⁵⁺ and MnTSPP³⁻ were used as control compounds of both high positive and negative charge where manganese was known to be in trivalent state. The singly charged manganese(III) salen was also prepared which was previously characterized as containing manganese in the trivalent state (Boucher, J. Inorg. Nucl. Chem. 36:531 (1974)). Finally, the magnetic susceptibility of Mn^{II}OBTM-4-PyP⁴⁺ was measured, the metal of which is in the divalent state (Batinic-Haberle et al, Arch. Biochem. Biophys. 343:225-233 (1997)). The magnetic moments are given in Table 3. In all cases, independently of the total charge of the compounds, the μ_{eff} are lower in methanol than in water. Based on those differences the magnetic moment of {Mn^{III}BVDME}₂ in water was predicted to be μ_{eff}=5.10 BM, which is indicative of manganese +3 oxidation state. The same oxidation state was previously established for a similar compound manganese(III) octaethylbilindione based on the magnetic moment in chloroform, μ_{eff}=4.7 BM (Balch et al, J. Am. Chem. Soc. 116:9114 (1994)).

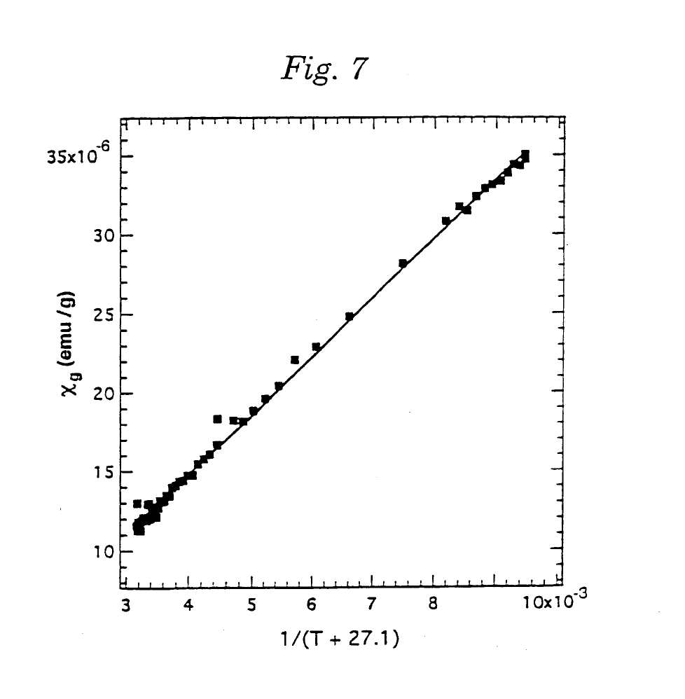

Magnetic Susceptibility in the Solid State. The measured gram susceptibilities (χ_{g}) of {Mn^{III}BVDME}₂ were independent of magnetic field strength from 300 to 3000 Oe at both 77 K and 286 K, indicating the absence of ferromagnetic impurities in the compound. Sixty χ_{g} values were obtained from 77 K to 286 K, and FIG. 7 shows the linear relationship between χ_{g} and 1/(T+27.1), in accord with the Curie-Weiss law (eq 3)

χ_{g}=C/(T+Θ)+χ_{d} [3]

The Weiss constant Θ was equal to −27.1, and the Curie constant C=(3.73±0.03)×10⁻³ emu/g/K. The diamagnetic gram susceptibility, χ_{d} obtained from the intercept was (−1.18±1.44)×10⁻⁷ emu/g. From the eq 4, where N is the number of manganese ions/g, 4 the unit Bohr

C=Nμ²β²/3k [4]

magnetron and k Boltzman constant, the magnetic moment μ was calculated to be 4.45 BM (Table 3). The Weiss constant of −27.1 K indicates antiferromagnetic interactions between the manganese centers in the solid. From the temperature-independent moment of 4.45 BM, and assuming “spin-only” behavior, it was calculated that 74% of the d⁴ Mn(III) ions are high-spin (S=2) (Physical Methods for Chemists, Drago, R. S., 2nd Ed., Saunders College Publishing. Ft. Worth (1977)), and the remaining 26% are in the low-spin (S=1) form.

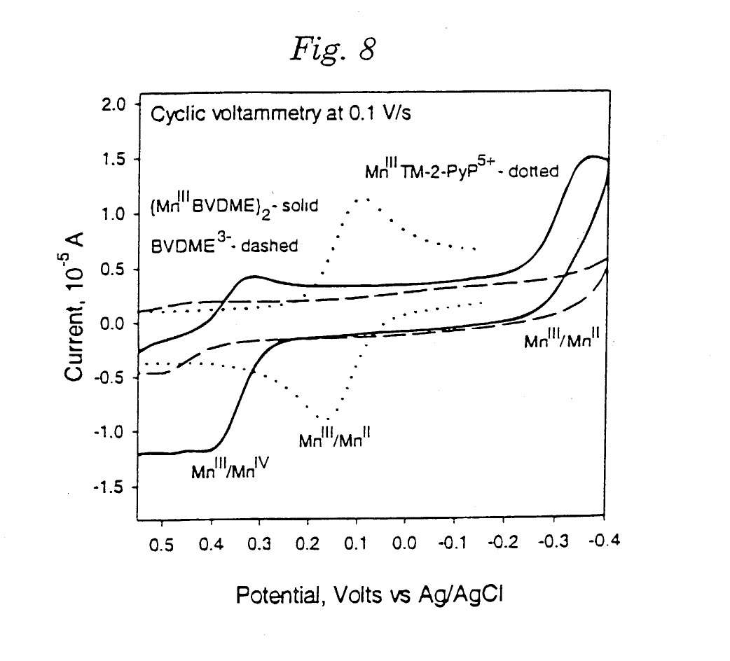

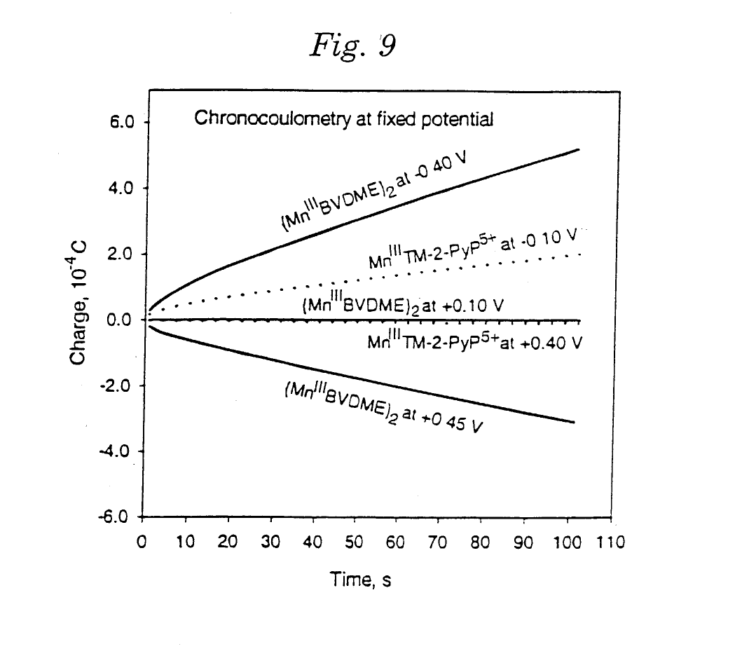

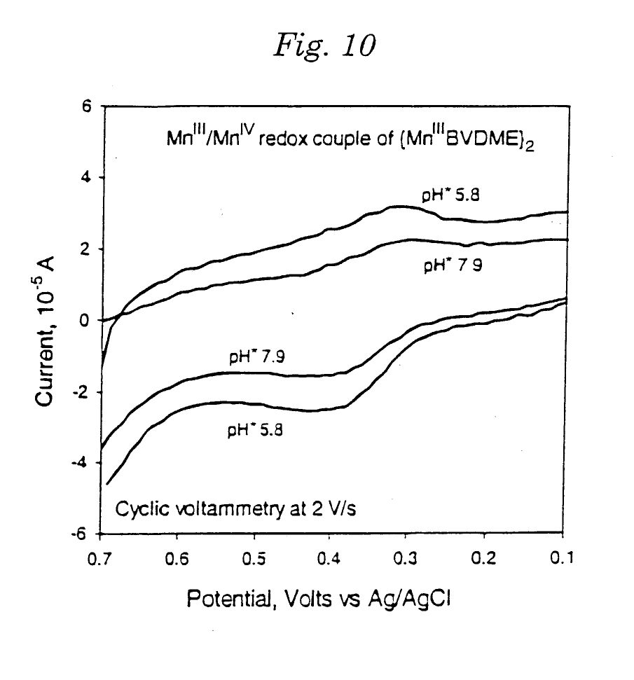

Electrochemistry. At 0.1 V/s scan rate and in the +0.6 to −0.4 V region, cyclic voltamrnetry of {Mn^{III}BVDME}₂ as compared to metal-free ligand BVDME³⁻, reveals two new waves, one reversible and the other one irreversible (FIG. 8). The irreversible wave seen at more negative potentials, becomes reversible at higher scan rates (see below). The magnetic moment μ_{eff}=5.10 BM, shown in Table 3, established +3 (Physical Methods for Chemists, Drago, R. S., 2nd Ed., Saunders College Publishing. Ft. Worth (1977)) as the stable oxidation state of both metal centers in the {Mn^{III}BVDME}₂ in the solution. The +3 oxidation state was confirmed by chronocoulometric measurements at constant potential (FIG. 9). No redox process was detected at +100 mV vs Ag/AgCl (between the two metal-centered waves) while oxidation takes place at potentials more positive than one wave (+0.45 V vs Ag/AgCl) and reduction takes place at potentials more negative than another wave (−0.40 V vs Ag/AgCl). For comparison, an electrochemically well-characterized manganese porphyrin Mn^{III}TIM-2-PyP⁵⁺ was used which, as expected, was reduced at −0.10 V vs Ag/AgCl but exhibited no electrochemistry at +0.40 V vs Ag/AgCl (FIG. 9). The voltammogram at +0.36 V vs Ag/AgCl is therefore ascribed to the Mn(III)/Mn(IV) couple, and the voltammogram at −0.32 V vs Ag/AgCl to Mn(III)/Mn(II) couple. The voltammograms obtained have peak-to-peak separation of 59 mV or higher which means that the redox processes at two metal centers in the dimeric {Mn^{III}BVDME}₂ complex occur independently. The reversible Mn(III)/Mn(IV) couple, that was found to be proton-independent (FIG. 10), could be described by eq 5:

1/2{Mn^{III}BVDME⁰}₂<===>1/2{Mn^{III}BVDME⁺}₂+e⁻ [5]

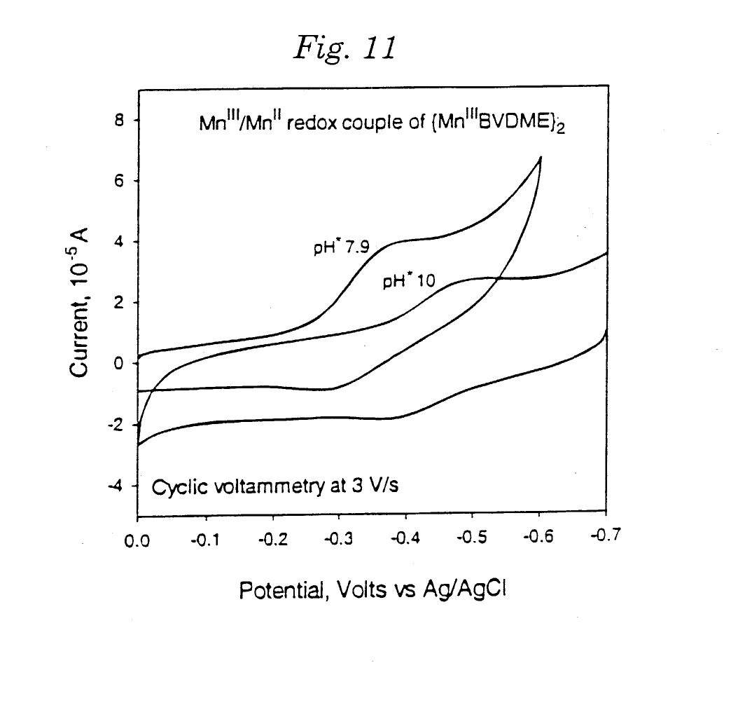

The quasi-reversible Mn(III)/Mn(II) couple was found to be proton-dependent (FIG. 11) and could be best presented by eq 6:

1/2{Mn^{III}BVDME⁰}₂+H⁺+e⁻<===>Mn^{II}HBVDME⁰ [6]

The Nernst equation applied to a one-proton (per metal center), one-electron redox reaction, predicts a change in the redox potential of −59 mV per pH unit (Astruc, Electron Transfer and Radical Processes in Transition-Metal Chemistry, pp 162, VCH Publishers. New York (1955)). Accordingly, a shift of −115 mV was observed when the pH* was increased from 7.9 to 10.0. Once the manganese is reduced to +2 state the enolic oxygen binding is no longer favored, and the dimeric structure either partially opens or falls apart accompanied by the protonation of the enolic oxygen. Thus the electrochemical reversibility was only achieved at high scan rates (3 V/s to 10 V/s) as shown in FIG. 11.

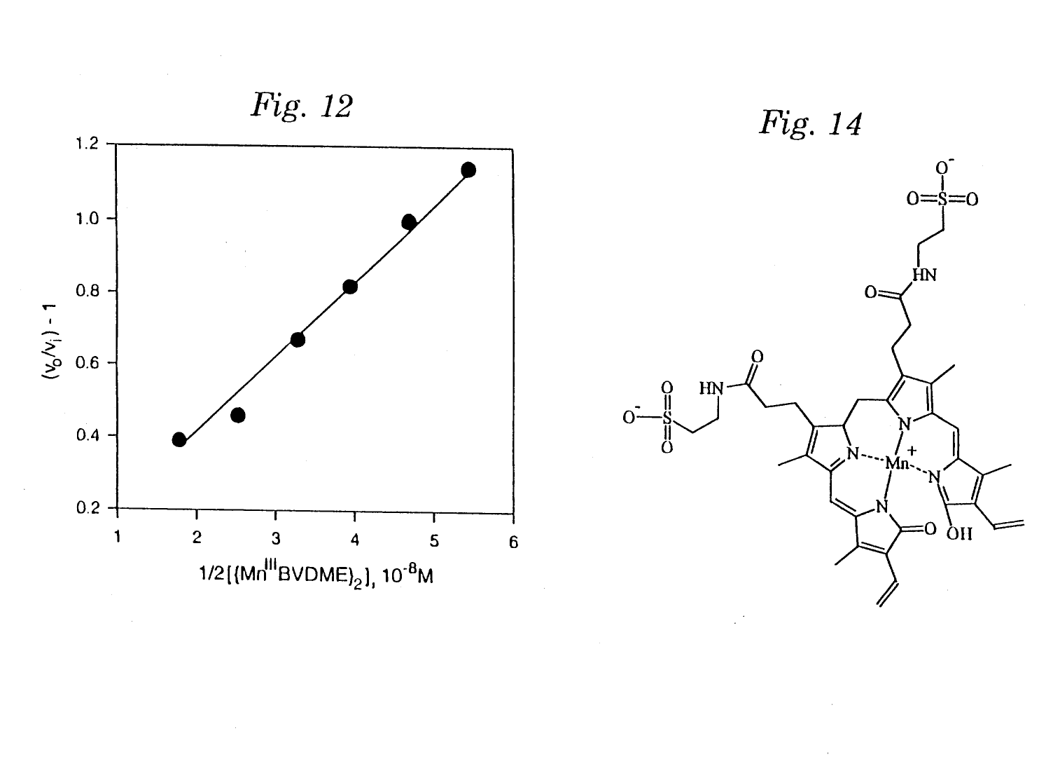

Dismutation of superoxide anion, O₂⁻. Assays were conducted in 0.05 M aqueous phosphate buffer, pH 7.8, 0.1 mM EDTA, ±15 μg/mL of catalase, ±albumin. Rate constants for the reaction with O₂⁻were based upon the competition of the compounds with cytochrome c. Neither interference with reaction of xanthine with xanthine oxidase, nor reoxidation of cytochrome c by manganese complex was observed. Neither biliverdin nor bilirubin showed any observable SOD-like activity which is consistent with previous study of Robertson and Fridovich (Robertson et al, Arch. Biochem. Biophys. 213:353 (1982)). When added at 1:1, albumin to compound ratio, no negative effect of albumin was observed. At higher albumin to compound ratios the SOD-like activity decreases and is 50% lower when the ratio becomes 3. Typically 1 μM stock methanolic solutions of {Mn^{III}BVDME}₂ were diluted to nM levels in the aqueous assay solution. From the plot {(v₀/vᵢ)−1} vs ½[{Mn^{III}BVDME}₂] (FIG. 12), based upon the competition (Sawada et al, Biochem. Biophys. Acta 327:257 (1973)) with 10 μM cytochrome c (k_{cyt}=2.6×10⁵ M⁻¹ s⁻¹) (Buttler et al, J. Biol. Chem. 257:10747 (1982)), it was calculated (per manganese) that IC₅₀=4.7×10⁻⁸ M and k_{cat}=5.5×10⁷ M⁻¹s⁻¹ at 25° C. (Table 4). The SOD-like activity equals that of Mn^{III}TM(E)-2-PyP⁵⁺ (Batinic-Haberle et al, Inorg. Chem. 38:4011 (1999), Kachadourian et al, Inorg. Chem. 38:391 (1999)).

The catalytic, SOD-like behavior of Mn^{III}salen⁺ was also studied and was observed only in the absence of chelator. EDTA (Liu et al, Arch. Biochem. Biophys. 315:74 (1994)). In the presence of chelator the metal-centered reduction was accompanied by loss of manganese.

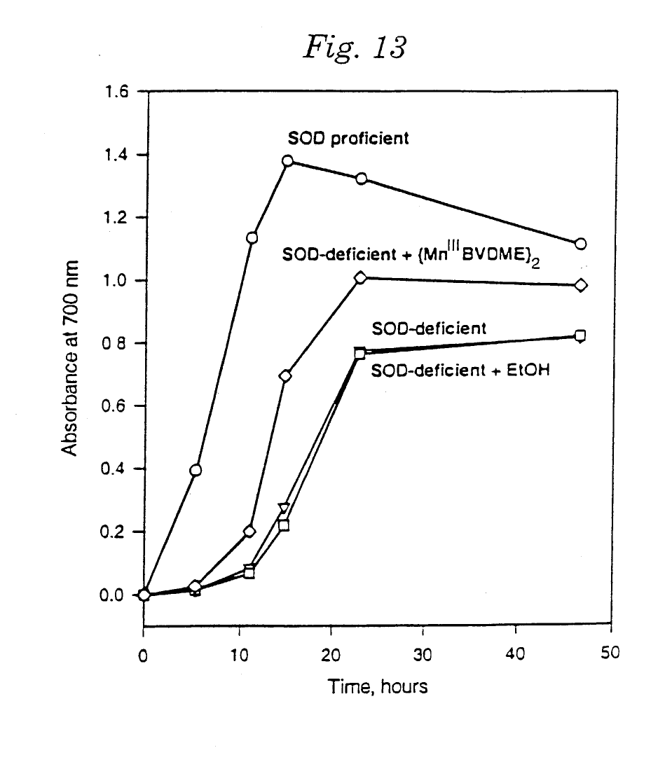

Effects on E coli. When {Mn^{III}BVDME}₂ is added to the minimal medium at a concentration of 0.1 to 13 μM the growth of SOD-deficient E. coli was markedly improved. After 15 hours of growth the SOD-proficient E. coli achieved 100% of its growth, SOD-deficient only 16%, but 50% in the presence of 13 μM (FIG. 13). The ligand itself, BVDME³⁻ was neither beneficial nor toxic and the Mn(II) complex did not affect the growth of the SOD-proficient E. coli.

### EXAMPLE 2

Two metallotetrapyrroles were tested for antioxidant activity, in vitro. In particular, the compounds were assayed for their ability to dismutate superoxide in the indirect method that utilizes xanthine and xanthine oxidase to generate superoxide and cytochrome c reduction as indicator of superoxide flux. Both compounds could dismutate superoxide with the MnBVDME being more potent than the MnBRDT (manganese (III) bilirubin ditaurate—see FIG. 14). The compounds were also assayed for their ability to prevent lipid peroxidation of rat brain homogenates by iron and ascorbate. The mixture of iron and ascorbate generates reactive oxygen species that oxidize biological lipids and these oxidized lipids can be detected as species that react with thiobarbituric acid (TBARS). Both compounds could inhibit the formation of TBARS and this ability correlated with their superoxide dismutase (SOD) activity (Table 5).

TABLE 5
Antioxidant activity of two metallotetrapyrroles

| | SOD Activity | Lipid Peroxidation |
|---|---|---|
| Compound | (USOD/mg)^{a} | (IC₅₀ μM)^{b} |
| MnBRDT | 6.2 | 70 |
| MnBVME | 10,700 | 0.3 |

a) SOD activity determined using a xanthine oxidase/cytochrome c assay

b) Lipid peroxidation was initiated in rat brain homogenates with iron/ascorbate and the IC₅₀ is the concentration of compound that reduced lipid peroxidation by one-half.

### EXAMPLE 3

When R₁ and R₆ of the compound of Formula I are propionate, the Mn^{III} Biliverdin (Mn^{III}BV)₂ dimer is formed. Its structure is the same as that of its dimethyl ester derivative (FIG. 2C). The same is true for its SOD activity, IC₅₀=4.7×10⁻⁸M. However, this compound lacks adequate stability in water solution.

All documents cited above are hereby incorporated in their entirety by reference.

One skilled in the art will appreciate from a reading of this disclosure that various changes in form and detail can be made without departing from the true scope of the invention.

TABLE 1

| Compound | R₁ | R₂ | R₃ | R₄ | R₅ | R₆ | R₇ | R₈ | α | β | γ | δ |
|---|---|---|---|---|---|---|---|---|---|---|---|---|
| Protoporphyrin | M | V | M | V | M | P | P | M | H | H | H | H |
| Mesoporhyrin | M | E | M | E | M | P | P | M | H | H | H | H |
| Deutero | M | H | M | H | M | P | P | M | H | H | H | H |
| porphyrin | | | | | | | | | | | | |
| Haemato- | M | B | M | B | M | P | P | M | H | H | H | H |
| porphyrin | | | | | | | | | | | | |

M = —CH₃
E = —CH₂CH₃
V = —CH═CH₂
P = —CH₂CH₂COOH, —CH₂CH₂COOR, —CH₂CH₂CO(NRR), —CH₂CH₂CO-sugar (sugar = glycerol, glucose, glucuronate etc.).

TABLE 2
Electrospray Mass Spectrometry Data of the 600 μM Methanolic
Solution of Manganese(III) Bilivercin Dimethylester. The Peaks Listed
were Obtained in the Range of Cone Voltages from 30 V to 180 V.
The Peaks that Correspond to the Isotopic Pattern are not Given.

| m/z | Species |
|---|---|
| 609 | H₂BVDME⁻ |
| 611 | H₄BVDME⁻ |
| 662^{a} | Mn^{IV}BVDME⁻ |
| 663^{b} | Mn^{III}HBVDME⁻ |
| 674 | (Mn^{III}HBVDME⁻ − Mn^{III}NaBVDME⁻)⁻⁻ |
| 685^{c} | Mn^{III}NaBVDME⁻ |
| 693 | (Mn^{III}NaBVDME⁻ − Mn^{III}KBVDME⁻)⁻⁻ |
| 695 | Mn^{III}HBVDME⁻ − CH₃OH |
| 701 | Mn^{III}KBVDME⁻ |
| 994 | ({Mn^{III}BVDME · Mn^{III}HBVDME⁻} − Mn^{III}HBVDME⁻)^{--d} |
| 1005 | ({Mn^{III}BVDME · Mn^{III}HBVDME⁻} − Mn^{III}NaBVDME⁻)^{--d} |
| 1017 | ({Mn^{III}BVDME · Mn^{III}NaBVDME⁻} − Mn^{III}NaBVDME⁻)^{--d} |
| 1274 | (H₄BVDME⁻ − Mn^{II}HBVDME)⁻ |
| 1324 | {Mn^{IV}BVDME⁻ · Mn^{III}BVDME} |
| 1325 | {Mn^{III}BVDME · Mn^{III}HBVDME⁻} |
| 1347 | {Mn^{III}BVDME · Mn^{III}NaBVDME⁻} |
| 1987 | ({Mn^{IV}BVDME⁻ · Mn^{III}BVDME} − Mn^{III}BVDME)^{-e} |
| 1988 | ({Mn^{III}BVDME · Mn^{III}HBVDME⁻} − Mn^{III}HBVDME)^{-e} |
| 2010 | ({Mn^{III}BVDME · Mn^{III}NaBVDME⁻} − Mn^{III}BVDME)^{-e} |
| ^{a}doubly oxidized. ^{b}doubly protonated and ^{c}doubly sodiated dimers are buried under these signals. ^{d}the peaks at m/z 1987, m/z 1988 and m/z 2010 could as well be assigned to singly charged oxidized {Mn^{IV}BVDME⁻ · Mn^{III}BVDME · Mn^{III}BVDME}, protonated {Mn^{III}BVDME · Mn^{III}BVDME · Mn^{III}HBVDME⁻}, | |
| #and sodiated trimer Mn^{III}BVDME · Mn^{III}BVDME · Mn^{III}NaBVDME⁻}, respectively. | |

^{e}Also, the peaks at m/z 994, m/z 1005 and m/z 1017 may be assigned to double charged protonated {Mn^{III}HBVDME⁻ · Mn^{III}BVDME · Mn^{III}HBVDME⁻}, mixed protonated and sodiated {Mn^{III}HBVDME⁻ · Mn^{III}BVDME · Mn^{III}NaBVDME⁻} and sodiated trimer {Mn^{III}NaBVDME⁻ · Mn^{III}BVDME · Mn^{III}NaBVDME⁻}, respectively.

TABLE 3
Magnetic Moments for Various Metalloporphyrins, Mn^{III}salen⁻,
{Mn^{III}OEB}₂ and {Mn^{III}BVDME}₂ in
D₂O and CD₃OD Solutions Determined at 25° C.

| | magnetic moment, μ_{erf} (BM) | | | | |
|---|---|---|---|---|---|
| | | CDCl₃ | | | μ_{erf}(D₂O) − |
| Compound | CD₃OD | solid state | | D₂O | μ_{erf}(CD₃OD) |
| Mn^{III}salen⁻ | 4.92 | | | 5.35 | 0.43 |
| Mn^{III}TSPP³⁻ | 4.14 | | | 4.83.4.9^{b} | 0.69 |
| Mn^{III}TE-2-PyP⁵⁻ | 4.00 | | | 4.62 | 0.62 |
| Mn^{III}TM-2(4)-PyP⁵⁻ | 4.14(4.10) | | | 4.57(4.60) | 0.43(0.50) |
| Mn^{III}TPP⁻ | 4.10 | | | | |
| {Mn^{III}OEB}₂ | | 4.7^{c} | | | |
| {Mn^{III}BVDME}₂ | 4.57. | 5.15^{d} | 4.44 | 5.10^{e} | 0.53^{f} |
| Mn^{II}OBTM-4-PyP⁴⁻ | 5.10 | | | | |

^{a}Theoretical spin-only magnetic moments for manganese(III) and manganese(II) high-spin complexes with 4 and 5 unpaired electrons are 4.90 BM and 5.92 BM, respectively.⁴⁰
^{b}ref 64:
^{c}ref 39:
^{d}ref 19:
^{e}the predicted μ_{erf} in D₂O.
^{f}the average difference

TABLE 4
The Comparison of Electrochemical Data and
SOD Activities of {Mn^{III}BVDME}₂, Mn^{III}Tm-2-Pyp⁵⁻^{a} and
Mn^{III}salen⁻ Determimed at 25° C.

| | E₁₂, V vs NHE^{b} | | | |
|---|---|---|---|---|
| | (E₁₂, V vs Ag/ | IC₅₀, | | |
| Compound | AgCl)^{d} | M^{c} | k_{cat}5⁻M^{-e} | U mg^{f} |
| Mn^{III/II}TM-2-PyP⁵⁻⁴⁻ | −0.22^{a} (−0.12)^{d} | 4.3 × | 6.0 × 10^{7a} | 8.500^{a} |
| | | 10^{-6a} | | |
| ½ | −0.45^{e} (−0.36)^{d} | 4.7 × | 5.5 × 10⁷ | 10.700 |
| {Mn^{III/IV}BVDME^{0/-}}₂ | | 10⁻⁸ | | |
| ½ {Mn^{III}BVDME}₂/ | −0.23^{c} (−0.32)^{d} | | | |
| Mn^{II}HBVDME | | | | |
| Mn^{III/II}salen^{-/0} | −0.13^{e} (−0.23)^{d} | 1.3 × | 6.0 × 10^{5f} | 700^{f} |
| | | 10^{-6f} | | |
| ^{a}Ref 4. | | | | |
| ^{b}In aqueous solution (pH 7.8, 0.1 M NaCl). | | | | |
| ^{c}IC₅₀ is the concentration that causes 50% of the inhibition of cytochrome c reduction by O₂⁻ in 0.05 M phosphate buffer, pH 7.8 at 25° C. 1 unit of SOD activity (U) is the quantity of the compound (mg) per 3.0 mL that caused 50% of the inhibition of the reduction of 10 μM cytochrome c by O₂⁻ produced at rate of 1.2 mM per minute. IC₅₀ k_{cat} and specific activities (U/mg) are calculated per | | | | |
| # manganese, i.e. per ½ {Mn^{III}BVDME}₂. | | | | |

^{d}In 90/10 (v/v) methanol/aqueous solution, pH* = 7.9 vs. Ag/AgCl.
^{e}The extrapolated redox potential in aqueous solution.
^{f}No SOD-like activity was observed in the presence of EDTA.

What is claimed is:

1. A compound of formula 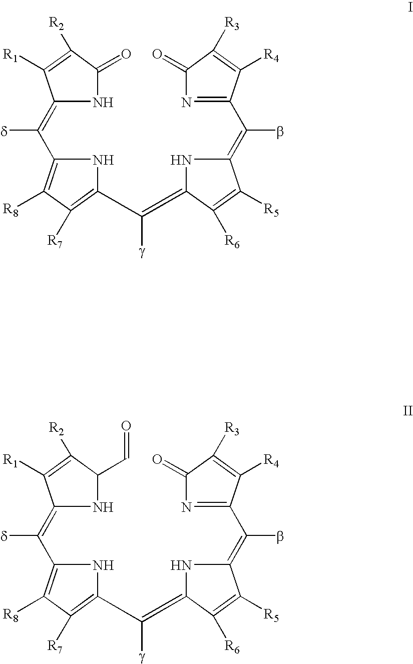
or pharmaceutically acceptable salt thereof,
wherein
R₁ through R₈ are, independently, —H, alkyl, 2-hydroxyalkyl, methoxyalkyl, halogen, nitro, cyano, trialkylammonium, formyl, amide of carboxylic acid, alkyl ester of carboxylic acid, carboxylic acid, glucuronyl or glyceryl ester of carboxylic acid, 1,2-dihydroxyalkyl, acetyl, vinyl, glycosyl or, taurate, and
β, γ and δ are, independently, —H, acetyl, glycyl, benzoate, phenylsulfonate, 2-, or 3-, or 4N-alkyl-pyridyl, nitrophenyl, halophenyl, methoxyalkyl, halogen, nitro, cyano, trialkylammonium, formyl, amide of carboxylic acid
with the proviso that when said compound is of formula I, β, γ and δ are —H, and said compound is not complexed with a metal, then R₁—R₈ are not methyl, vinyl, methyl, vinyl, methyl, propionic acid, propionic acid and methyl, respectively.

2. A compound of formula 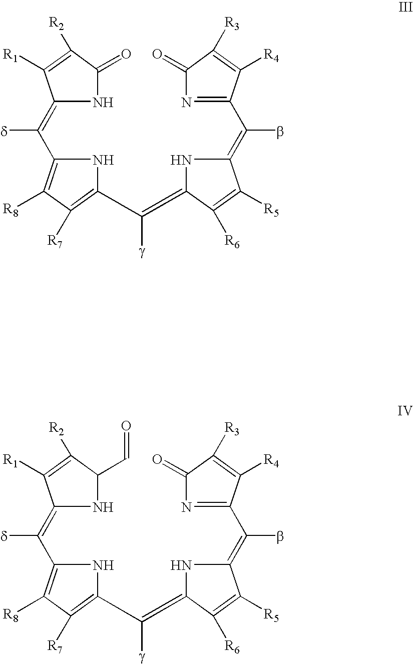
or pharmaceutically acceptable salt thereof,
wherein
R₁ through R₈ are, independently, —H, alkyl, 2-hydroxyalkyl, methoxyalkyl, halogen, nitro, cyano, trialkylammonium, formyl, amide of carboxylic acid, alkyl ester of carboxylic acid, carboxylic acid, glucuronyl or glyceryl ester of carboxylic acid, 1,2-dihydroxyalkyl, acetyl, vinyl, glycosyl or, taurate, and
β, γ and δ are, independently, —H, acetyl, glycyl, benzoate, phenylsulfonate, 2-, or 3-, or 4-N-alkyl-pyridyl, nitrophenyl, halophenyl, methoxyalkyl, halogen, nitro, cyano, trialkylammonium, formyl, amide of carboxylic acid
with the proviso that when said compound is of formula III, β, γ and δ are —H and said compound is not complexed with a metal, then R₁-R₈ are not methyl, vinyl, methyl, vinyl, methyl, propionic acid, propionic acid and methyl, respectively.

3. The compound according to claim 1 or 2 wherein R₁ through R₈ are, independently, —H, C₁-C₅ alky, 2-hydroxy C₁-C₅ alkyl, C₁-Csalkyl ester of C₁-C₅carboxylic acid, C₁-C₅carboxylic acid, glucuronyl or glyceryl ester of C₁-C₅carboxylic acid, 1,2-dihydroxy C₁-C₅ alkyl, acetyl, vinyl, glycosyl, taurate, chloro, fluoro, bromo, nitro, cyano, trimethylammonium, or formyl, and
β, γ and δ are, independently, —H, acetyl, glycyl, benzoato, phenylsulfonato, 2-, 3- or 4-N-C₁-C₅ alkyl-pyridyl, nitrophenyl, bromo-, chloro- or fluorophenyl or 2-, 3- or 4-N-C₁-C₅alkylsulfonatopyridyl.

4. The compound according to claim 1 or 2 wherein said compound is complexed with a metal selected from the group consisting of zinc, iron, nickel, cobalt, copper, manganese.

5. The compound according to claim 4 wherein said compound is complexed with manganese.

6. A dimeric form of the compound according to claim 4.

7. The dimer according to claim 6 wherein said metal is manganese.

8. The dimer according to claim 7 wherein said dimer is of the formula:

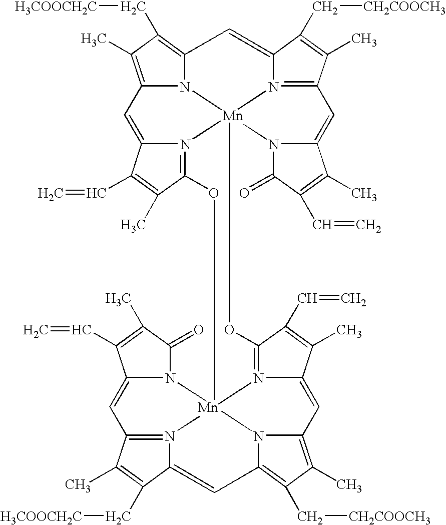

9. A method of protecting cells from oxidant-induced toxicity comprising contacting said cells with a protective amount of the compound according to claim 1 or 2 so that said protection is effected.

10. The method according to claim 9 wherein said compound is complexed with a metal selected from the group consisting of manganese, iron, copper, cobalt, nickel or zinc.

11. The method according to claim 10 wherein said metal is manganese.

12. The method according to claim 11 wherein said cells are mammalian cells.

13. The method according to claim 12 wherein said cells are cells of an isolated organ.

14. The method according to claim 13 wherein said cells are cells of an organ transplant.

15. A method of treating a patient suffering from a condition that results from or that is exacerbated by oxidant-induced toxicity comprising administering to said patient an effective amount of the compound according to claim 1 or 2 so that said treatment is effected.

16. The method according to claim 15 wherein said compound is complexed with a metal selected from the group consisting of manganese, iron, copper, cobalt, nickel or zinc.

17. The method according to claim 16 wherein said compound is complexed with manganese.

18. A method of treating a pathological condition of a patient resulting from the production or accumulation of a degradation product of NO or a biologically active form thereof, comprising administering to said patient an effective amount of the compound according to claim 1 or 2 so that said treatment is effected.

19. The method according to claim 18 wherein said compound is complexed with a metal selected from the group consisting of manganese, iron, copper, cobalt, nickel or zinc.

20. The method according to claim 19 wherein said compound is complexed with manganese.

21. A method of treating a patient for an inflammatory disease comprising administering to said patient an effective amount of the compound according to claim 1 or 2 so that said treatment is effected.

22. The method according to claim 21 wherein said compound is complexed with a metal selected from the group consisting of manganese, iron, copper, cobalt, nickel or zinc.

23. The method according to claim 22 wherein said compound is complexed with manganese.

24. A method of treating a patient for an ischemic reperfusion injury comprising administering to said patient an effective amount of the compound according to claim 1 or 2 so that said treatment is effected.

25. The method according to claim 24 wherein said compound is complexed with a metal selected from the group consisting of manganese, iron, copper, cobalt, nickel or zinc.

26. The method according to claim 25 wherein said compound is complexed with manganese.

27. The method according to claim 24 wherein said ischemic reperfusion injury results from a stroke.
